(19) United States
(12) Patent Application Publication
(10) Pub. No.: US 2017/0304156 A1
(43) Pub. Date: Oct. 26, 2017

(54) FORMULATION

(71) Applicants: UNIVERSITY OF LEEDS, Leeds (GB); University Court of the University of St Andrews, St Andrews, Fife (GB)

(72) Inventors: Animesh JHA, Leeds (GB); Mandeep Singh DUGGAL, Leeds (GB); Billy Donald Orac RICHARDS, Leeds (GB); Antonios ANASTASIOU, Leeds (GB); Christian Thomas Alcuin BROWN, St Andrews (GB); Wilson SIBBETT, St Andrews (GB)

(73) Assignees: UNIVERSITY OF LEEDS, Leeds (GB); University Court of the University of St Andrews, St Andrews, Fife (GB)

(21) Appl. No.: 15/514,058

(22) PCT Filed: Sep. 4, 2015
(86) PCT No.: PCT/GB2015/052557

§ 371 Date: Mar. 24, 2017

(30) Foreign Application Priority Data
Sep. 26, 2014 (GB) 1417020.3

(51) Int. Cl. A61K 6/00; A61K 6/033; A61L 27/56; A61K 6/04; A61L 27/12; A61K 6/093; A61L 27/18; A61L 27/52

(52) U.S. Cl. CPC A61K 6/0017; A61L 27/18; A61K 6/033; A61L 27/56; A61L 27/52; A61K 6/0067; A61L 27/12; A61K 6/093; A61K 6/043; A61L 2430/02; A61L 2430/12

(57) ABSTRACT

The present invention relates to a hydrogel formulation in which the solid phase is composed of a continuous net work of siloxane bonds and one or more calcium phosphate phases doped with one or more metal dopants.

The present invention relates to a hydrogel formulation, to a process for preparing the hydrogel formulation, to its use in combating (eg treating or preventing) a dental condition, whitening or veneering a tooth or generating an image of an exposed dentinal surface of a tooth and to a cast structure composed of the sintered hydrogel formulation.

There is growing interest in materials which can assist in bone and teeth regeneration especially in developed countries where higher life expectancy has increased the prevalence of age-related conditions such as osteoporosis and tooth loss due to periodontal disease, trauma and dental caries. Furthermore in developed countries, there is a significant clinical need to address tooth hypersensitivity which is caused by the loss of enamel and exposure of dental tubules. Hypersensitivity manifests as pain when the teeth are exposed to hot or cold temperatures. Most treatments only address the symptoms or temporarily occlude the tubules to prevent the exposure of the stimulus to the dental nerves. Synthetic hydroxyapatite (HAp) is used for this biomedical purpose due to its close resemblance to naturally occurring hydroxyapatite. A coating of synthetic hydroxyapatite covers the dentine and restores the enamel surface but the conditions that must be fulfilled for its optimal functioning are stringent. The materials must be closely matched to the properties of the tooth and bond to the surface whilst being non-toxic and displaying good biocompatibility. WO-A-2012/046082 describes the preparation of HAp by hydrothermal or chemical precipitation synthesis.

The present invention is based on the recognition that by co-precipitating doped calcium phosphate (CaP) phases concurrently with the hydrolysis and polycondensation of silicon alkoxides or silanols there is formed a hydrogel formulation which exhibits rapid densification on exposure to laser irradiation (eg low energy laser irradiation by (for example) PLD).

Thus viewed from one aspect the present invention provides a hydrogel formulation comprising:
a solid phase composed of a continuous network of siloxane bonds and one or more calcium phosphate phases doped with one or more metal dopants; and
an aqueous phase,
   or a precursor or anhydrate thereof.

The hydrogel formulation of the invention advantageously provides a means for rapid homogeneous casting of biocompatible calcium phosphate phases in situ during tissue engineering. A layer of the hydrogel formulation is sinterable into a smoother and more pristine surface than is achievable by calcium phosphate material alone.

The hydrogel formulation may be a dispersion in which the solid phase is continuous and the aqueous phase is discontinuous.

The continuous network of siloxane bonds may be a continuous polymer network. The continuous polymer network may be a chain-like continuous polymer network. The continuous polymer network may be a 1-, 2- or 3-dimensional continuous polymer network. The continuous network may be a covalent polymer network.

Preferably the aqueous phase is water.

The hydrogel formulation or anhydrate thereof is typically physiologically tolerable. The hydrogel formulation or anhydrate thereof may be osteogenetic, osteoconductive or osteoinductive.

The precursor may be a colloid (eg a sol). The anhydrate may be obtainable by heating the hydrogel formulation (eg by calcining, ablating or sintering such as photosintering the hydrogel formulation). The anhydrate may be crystalline or amorphous and take the form of a paste, powder or spray.

The (or each) metal dopant may be ions of an alkaline earth metal, a rare earth element, a transition metal or aluminium.

In a preferred embodiment, the one or more metal dopants is or includes ions of a rare earth element.

Photosensitization of the one or more calcium phosphate phases with ions of a rare earth element facilitates the efficient absorption of laser energy and promotes rapid ablation. This may be exploited to give efficient packing and sintering during tooth filling. Photosensitization minimises collateral damage of healthy tissue by keeping the temperature low (eg below 41° C.).

Preferably the ions of the rare earth element exhibit absorption bands which substantially match or overlap one or more absorption bands of the one or more calcium phosphate phases. Particularly preferably the ions of the rare earth element exhibit absorption bands which substantially match or overlap one or more absorption bands of one or more of the OH⁻ ion, CO₃²⁻ ion, phosphate ion or water (eg the first harmonic of the phosphate band or the fundamental OH band).

Preferably the ions of the rare earth element exhibit absorption bands in or overlapping the range 1400 to 1800 nm.

Preferably the ions of the rare earth element exhibit absorption bands in or overlapping the range 2700 to 3500nm.

Preferably the ions of the rare earth element exhibit absorption bands in or overlapping the range 4000 to 4500 nm.

Preferably the ions of the rare earth element exhibit absorption bands in or overlapping the range 1900 to 2100 nm.

Preferably the ions of the rare earth element and calcium have a substantially similar radius (eg an ionic radius within ±15%). This advantageously facilitates the substitution of calcium by the rare earth ion.

The (or each) metal dopant may usefully exhibit absorbtion or excitation at (for example) desirable wavelengths (for example UV or visible wavelengths). The (or each) metal dopant may exhibit broad absorbtion bands which can be exploited to minimise heat dissipation during photosintering and enhance safety.

The rare earth element may be a lanthanide. The rare earth element may be selected from the group consisting of cerium, gadolinium, holmium, thulium, dysprosium, erbium, ytterbium and neodymium.

Preferably the rare earth element is selected from the group consisting of dysprosium, cerium, ytterbium, erbium, holmium and thulium. Particularly preferably the rare earth element is selected from the group consisting of erbium, cerium and ytterbium.

The rare earth element may be present in an amount relative to the one or more calcium phosphate phases in excess of 100 ppm (eg in the range 100 to 50000 ppm).

The one or more metal dopants may be or include ions of a transition metal. The one or more metal dopants may be or include ions of iron, chromium or silver.

The one or more metal dopants may be or include ions of an alkaline earth metal. The one or more metal dopants may be or include ions of barium or strontium.

Preferably the one or more metal dopants is or includes aluminium ions. Aluminium ions have a strong tendency to form aluminium phosphate at the expense of OH and HPO₄ ions which means that the formation of carbonate via bicarbonate at OH sites (which is the cause of weak bonding in the lattice) is reduced. Thus the hydrogel formulation when sintered exhibits an enhanced ability to withstand acid attack in the oral environment.

In a preferred embodiment, the one or more calcium phosphate phases are fluoride ion-substituted. Substitution of hydroxide ions by fluoride ions in the one or more calcium phosphate phases advantageously redresses the charge imbalance caused by the substitution of calcium (2+) with a rare earth ion (3+).

Preferably the one or more calcium phosphate phases includes fluorapatite.

In a preferred embodiment, the one or more calcium phosphate phases are fluoride ion-substituted and the one or more metal dopants is or includes aluminium ions.

In a preferred embodiment, the one or more calcium phosphate phases are fluoride ion-substituted and the one or more metal dopants is or includes ions of a rare earth element.

In a particularly preferred embodiment, the one or more calcium phosphate phases are fluoride ion-substituted and the one or more metal dopants is or includes aluminium ions and ions of a rare earth element.

The one or more calcium phosphate phases may be present in the hydrogel formulation in an amount in excess of 50 wt % (eg in the range 50 to 70 wt %).

Preferably the one or more calcium phosphate phases is or includes synthetic hydroxyapatite (Ca₁₀(PO₄)₆(OH)₂) or a synthetic precursor thereof. The synthetic hydroxyapatite may be nanocrystalline. The synthetic precursor of synthetic hydroxyapatite may be octacalcium phosphate.

Preferably the one or more calcium phosphate phases is or include a synthetic mineral of formula CaHPO₄.xH₂O(wherein x is 0, 1 or 2). Particularly preferably x is 0 or 2.

The (or each) metal dopant typically substitutes calcium in the crystal lattice of the synthetic hydroxyapatite (or synthetic precursor thereof) or of the one or more CaHPO₄.xH₂O minerals.

Typically the predominant phases of the one or more calcium phosphate phases are the one or more CaHPO₄.xH₂O minerals.

Preferably the one or more calcium phosphate phases include monetite. Monetite may be the predominant calcium phosphate phase.

Preferably the one or more calcium phosphate phases include brushite. Brushite may be the predominant calcium phosphate phase.

Preferably the one or more calcium phosphate phases include monetite and brushite.

The one or more calcium phosphate phases may be a solid solution of synthetic hydroxyapatite, brushite and monetite.

The solid phase of the hydrogel formulation may be further composed of one or more phases of the source of metal dopant or fluoride ions. The source of metal dopant or fluoride ions may be one or more of the group consisting of calcium fluoride, stannous fluoride, aluminium chloride and aluminium phosphate. Preferably the solid phase of the hydrogel formulation is further composed of CaF₂ and AlPO₄.

Preferably the solid phase of the hydrogel formulation is further composed of chitosan.

The one or more calcium phosphate phases may be nanoparticulate. For example, the nanoparticles may be substantially flat, substantially rod-like, platelets, flakes, needles or whiskers.

Preferably the hydrogel formulation (or the anhydrate thereof) is capable of at least partially (eg fully) occluding a dentinal tubule in a tooth (eg to a depth of at least 1 μm).

Preferably the hydrogel formulation (or the anhydrate thereof) is capable of at least partially (eg substantially fully) occluding a major proportion of the dentinal tubules in a tooth. The major proportion may be 80% or more, preferably 90% or more.

Preferably the hydrogel formulation (or the anhydrate thereof) is capable of substantially fully occluding a major proportion of the dentinal tubules in a tooth. The major proportion may be 60% or more, preferably 70% or more.

Preferably the hydrogel formulation is obtained or obtainable by a co-precipitation reaction carried out in the presence of a siloxane network precursor.

In a preferred embodiment, the hydrogel formulation is obtained or obtainable by a process comprising:
(a) preparing an aqueous mixture of a calcium ion-containing solution, a phosphate ion-containing solution and a metal dopant-containing solution in the presence of the siloxane network precursor;
(b) causing the formation of the solid phase in the aqueous mixture; and
(c) isolating the hydrogel formulation.

The siloxane network precursor may be a silanol or silicon alkoxide.

A preferred siloxane network precursor is a silicon tetralkoxide.

The siloxane network precursor may be selected from the group consisting of Si(OCH₃)₄, Si(OC₂H₅)₄, Si(OⁱPr)₄, Si(O^{t}Bu)₄ or Si(OⁿBu)₄.

A preferred siloxane network precursor is an orthosilicate. Particularly preferred are tetraethyl orthosilicate (TEOS), tetramethyl orthosilicate (TMOS) or tetrakis(2-hydroxyethyl) orthosilicate (THEOS). Most preferred is tetraethyl orthosilicate (TEOS).

A preferred siloxane network precursor has the formula:

R(R′)(R″)SiOR′″

wherein:
each of R, R′ and R″ is independently selected from hydrogen, a C₁₋₆-alkyl group or an optionally hydroxylated or alkoxylated Cₗ₋₆-alkoxy group; and
R′″ is hydrogen or an optionally hydroxylated or alkoxylated C₁₋₆-alkyl group.

Each of R, R′ and R″ may be independently selected from methyl, ethyl, propyl (eg isopropyl) or butyl (eg tert-butyl).

Preferably each of R, R′ and R″ is a C₁₋₆-alkoxy group.

Each of R, R′ and R″ may be independently selected from methoxy, ethoxy, propoxy (eg isopropoxy) and butoxy (eg tert-butoxy).

Preferably R′″ is a C₁₋₆-alkyl group.

R′″ may be selected from methyl, ethyl, propyl (eg isopropyl) or butyl (eg tert-butyl).

Typically each of R, R′ and R″ is the same. Preferably each of R, R′, R″ and OR′″ is the same.

The source of the calcium ions in the calcium ion-containing solution may be a calcium salt (eg a carbonate, nitrate or chloride salt). Typically the calcium salt is hydrated.

The source of the phosphate ions in the phosphate ion-containing solution may be a phosphate salt (eg a hydrogen phosphate salt). Typically the phosphate salt is hydrated.

Preferably the metal dopant-containing solution includes ions of a rare earth element. The source of ions of the rare earth element may be a salt such as a carbonate, acetate, hydroxide, nitrate, oxide or halide salt (preferably an acetate, citrate, nitrate, chloride, fluoride or chloride salt). The salt may be crystalline. The salt may be hydrated. The amount of the source of the ions of the rare earth element used to prepare the metal dopant-containing solution is preferably 5 wt % or less (particularly preferably 1 to 5 wt %, more preferably 1 to 2 wt %) of the total weight of the source of calcium ions used to prepare the calcium ion-containing solution and the source of the phosphate ions used to prepare the phosphate ion-containing solution. Specific examples of the source of the ions of the rare earth element include Tm(OH)₃, Er(OH)₃, Tm₂O₃, Yb₂O₃, Ho₂O₃, Er₂O₃, TmF₃, ErF₃, Ce(NO₃)₃.6H₂O, Tm(NO₃)₃.5H₂O, Er(NO₃)₃.5H₂O, Yb(NO₃)₃.5H₂O and ErCl₃.

Preferably the metal dopant-containing solution includes aluminium ions. The amount of the source of aluminium ions is preferably such that the aluminium ions are present in an amount of 5 wt % or less (particularly preferably 2 wt % or less) of the total weight of the source of the calcium ions used to prepare the calcium ion-containing solution and the source of the phosphate ions used to prepare the phosphate ion-containing solution. The source of aluminium ions may be an aluminium salt. For example the source of aluminium ions may be aluminium nitrate, aluminium phosphate or aluminium trichloride hexahydrate. Preferred is Al(NO₃)₃.9H₂O.

Preferably the aqueous mixture further includes fluoride ions. The amount of the source of fluoride ions is preferably 5 wt % or less (particularly preferably 2 wt % or less) of the total weight of the source of calcium ions used to prepare the calcium ion-containing solution and the source of the phosphate ions used to prepare the phosphate ion-containing solution. For example, the source of fluoride ions may be calcium fluoride, ammonium fluoride or stannous fluoride. Ammonium fluoride is preferred and advantageously promotes gelation.

Preferably the aqueous mixture further includes an acidic solution of chitosan.

The calcium ion-containing solution and the phosphate ion-containing solution in step (a) are preferably such that the molar ratio of Ca:P in the aqueous mixture is about 1.67. For example in step (a), the phosphate ion-containing solution may be added dropwisely to the calcium ion-containing solution until the molar ratio of Ca:P in the aqueous mixture is about 1.67.

In step (a), the aqueous mixture may be agitated (eg stirred). In step (b), the aqueous mixture may be agitated (eg stirred). Continuous agitation in steps (a) and (b) promotes homogeneity in the hydrogel formulation.

In step (a), the aqueous mixture may be aged (eg for 24 hours or more).

The pH of the aqueous mixture is typically 6 or less, preferably 5.5 or less, particularly preferably 5 or less, more preferably 4.5 or less. A lower pH promotes gelation.

Preferably step (a) is preceded by:
(a0) preparing an aqueous pre-mixture of a pair of the calcium ion-containing solution, the phosphate ion-containing solution, the metal dopant-containing solution, the siloxane network precursor and (when present) the source of fluoride ions.

Particularly preferably the pair includes the siloxane network precursor. More preferably the pair is the siloxane network precursor and calcium ion-containing solution.

Preferably step (a) is additionally preceded by:
(a1) preparing an additional aqueous pre-mixture of an additional pair or a triplet of the calcium ion-containing solution, the phosphate ion-containing solution, the metal dopant-containing solution, the siloxane network precursor and (when present) the source of fluoride ions.

The additional pair may be the source of ions of a rare earth element and the source of fluoride ions.

Of independent patentable significance is that the presence of one or more metal dopants serves to promote gelation in a co-precipitation reaction carried out in the presence of a siloxane network precursor.

Viewed from a further aspect the present invention provides a process for preparing a hydrogel formulation as hereinbefore defined comprising:
(a) preparing an aqueous mixture of a calcium ion-containing solution, a phosphate ion-containing solution and a metal dopant-containing solution in the presence of the siloxane network precursor;
(b) causing the formation of the solid phase in the aqueous mixture; and
(c) isolating the hydrogel formulation.

Steps (a), (b) and (c) may be as hereinbefore defined.

Viewed from a yet further aspect the present invention provides a method for combating (eg treating or preventing) a dental condition in a tooth of a human or non-human animal subject comprising:
(A) applying an amount of a hydrogel formulation as hereinbefore defined in which the one or more metal dopants is or includes ions of a rare earth element to the tooth to cause at least partial occlusion of dentinal tubules; and
(B) irradiating the amount of the hydrogel formulation with laser irradiation so as to promote densification.

The dental condition may be dental caries, tooth wear or decay, sensitivity (eg acute hypersensitivity) or pain attributable to carious infection.

Preferably in step (A) the amount of the hydrogel formulation is applied to an exposed dentinal surface.

Preferably the laser irradiation is eye-safe.

Preferably the laser irradiation is infrared irradiation. Particularly preferably the laser irradiation is near infrared, mid infrared or short wavelength infrared irradiation. More preferably the laser irradiation is short wavelength infrared irradiation

The wavelength of laser irradiation may be in the range 980 to 4500 nm (preferably 1400 to 3000 nm).

The wavelength of laser irradiation may be coincident with one or more absorption bands of the OH⁻ ion, CO₃²⁻ ion, phosphate ion or water.

Preferably the wavelength of laser irradiation is in the range 1400 nm to 1800 nm. A wavelength in the range 1400-1800 nm minimizes side-affects and allows (for example) more than one order of magnitude more pulse energy to be delivered to a subject whilst still retaining an eye-safe Class I classification according to the International Standard on the Safety of Laser Products (IEC 60825-1). A wavelength in the range 1400 to 1800 nm is advantageously coincident with the absorption bands of the OH⁻ ion first harmonic.

Preferably the wavelength of laser irradiation is in the range 2700 to 3500 nm. A wavelength in the range 2700 to 3000 nm is advantageously coincident with the fundamental OH⁻ ion absorbtion band and phosphate ion harmonics.

Preferably the wavelength of laser irradiation is in the range 1900 to 2100 nm. A wavelength in the range 1900 to 2100 nm is advantageously coincident with the absorption bands of the OH⁻ ion overtone and CO₃²⁻ harmonics.

Preferably the wavelength of laser irradiation is in the range 4000 to 4500 nm. A wavelength in the range 4000 to 4500 nm is advantageously coincident with the absorption bands of the OH⁻ ion and CO₂ harmonics.

The laser may be a continuous wave laser (eg a near IR continuous wave laser). The laser may be a pulsed laser (eg an ultrashort pulsed laser). The laser may generate ultrashort pulses. The pulsed laser may be a pico, nano, micro or femtosecond pulsed laser. The laser may emit pulses of a length in the range 20 fs to 150 ps (eg about 135 ps). Preferably the pulsed laser is a femtosecond pulsed laser.

The laser may be (for example) a CO₂ laser, an Er-doped or Ho-doped Nd-YAG laser, a Tm-doped laser, a Ti-sapphire laser, a diode pumped laser (such as a Yb-doped or Cr-doped crystal laser) or a fibre optic laser.

The laser may be a short pulsed fibre laser in which the power is delivered (for example) using a silica fibre.

The pulse energy is typically in the range 1 nl to 1 μJ. The pulses may be emitted with a repetition rate up to 10 GHz (eg 100 MHz). The average power of the laser may be sub-Watt.

Typically the laser irradiation is a laser beam focussed to a relatively small spot size (for example about 30 μm). The laser beam may have a Gaussian or non-Gaussian shape.

Preferably the laser beam has a non-Gaussian shape. Examples of laser beams having a non-Gaussian shape include an Airy beam, Laguerre-Gaussian beam or Bessel beam.

Particularly preferably the laser beam is a Bessel beam. In this embodiment, a non-diverging centre beam is surrounded by rings. Radiation propagates in the central region with the diffractive behaviour associated with Gaussian propagation allowing a fixed width beam to be maintained over much longer distances. This relaxes the strict alignment otherwise needed to obtain successful sintering. Furthermore the reconstructive behaviour of the Bessel beam advantageously allows it to be used in environments where contamination may cause scattering to an equivalent Gaussian beam.

The advantages of the hydrogel formulation of the invention may be further exploited in methods which are solely cosmetic (non-restorative).

Viewed from a still yet further aspect the present invention provides a cosmetic method for whitening or veneering a tooth of a human or non-human animal subject comprising:
(1) applying an amount of a hydrogel formulation as hereinbefore defined to a surface of the tooth other than a dentinal surface; and
(2) irradiating the amount of the hydrogel formulation with laser irradiation so as to promote densification.

Preferably in step (1) the hydrogel formulation is applied solely to the enamel surface of the tooth.

A rare-earth ion emits radiation when excited at an absorption wavelength. In a further patentable aspect, the present invention is able to capture the emitted light to generate an image of (for example) a dental cavity into which a hydrogel formulation of the invention has been administered.

Viewed from an even still yet further aspect the present invention provides a method for generating an image of an exposed dentinal surface of a tooth of a human or non-human animal subject comprising:
(A) administering an amount of a hydrogel formulation as hereinbefore defined in which the one or more metal dopants is or includes ions of a rare earth element to the exposed dentinal surface;
(B) irradiating the hydrogel formulation with irradiation;
(C) capturing the radiation emitted by the hydrogel formulation ; and
(D) generating from the radiation emitted by the hydrogel formulation an image of the exposed dentinal surface.

The rapid provision of an image of the site of administration provides (for example) information on the state of crystallisation of the rare earth ion-containing metal dopant, the structure and morphology of the hydrogel formulation or the health of the dentine.

The exposed dentinal surface may be a part of a dental cavity or a characteristic of dental caries.

Steps (B), (C) and (D) may be carried out spectroscopically. Steps (B), (C) and (D) may be carried out by IR, Raman or fluorescence spectroscopy.

Viewed from an even still further aspect the present invention provides a self-supporting structure (eg a biomineral structure) composed of a sintered (eg laser-sintered) or ablated hydrogel formulation as hereinbefore defined.

The superficial and bulk strength of the self-supporting structure together with its porosity may be controlled by sintering to achieve the conditions essential for osteoinduction, osteoconduction and osseointegration. The self-supporting structure may be bespoke bone material in which the collagen, growth factor and mesenchymal stem cells may be cultured ex vivo prior to implantation as a xenograft.

The self-supporting structure may be a xenograft, bone graft, implant (eg dental implant), transplant or enamel replacement.

The self-supporting structure may be a cast structure. The self-supporting structure may be a mineral or composite structure.

Viewed from a furthest aspect the present invention provides the use of the hydrogel formulation as hereinbefore defined in restorative or cosmetic dentistry or in 3D printing.

The present invention will now be described in a non-limitative sense with reference to Examples and the Figures in which:

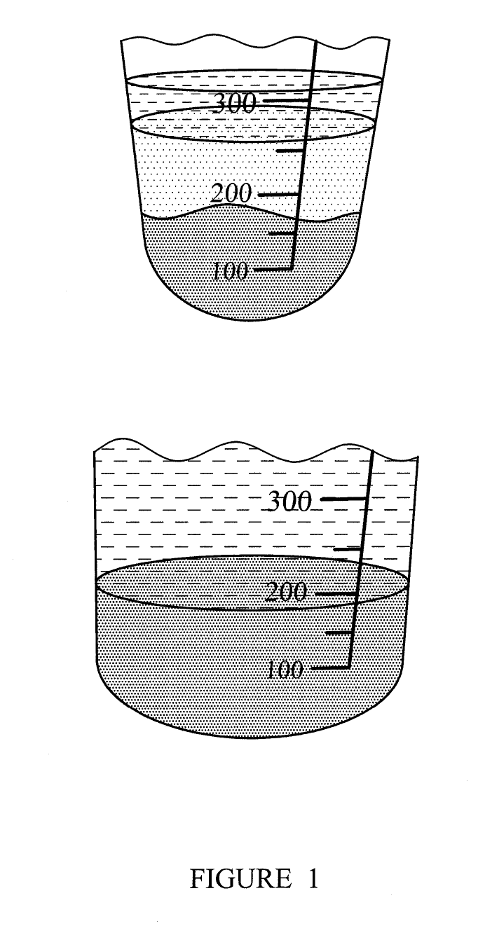

FIG. 1 is an image of the CaP gels present in (a) batch 1 and (b) batch 2;

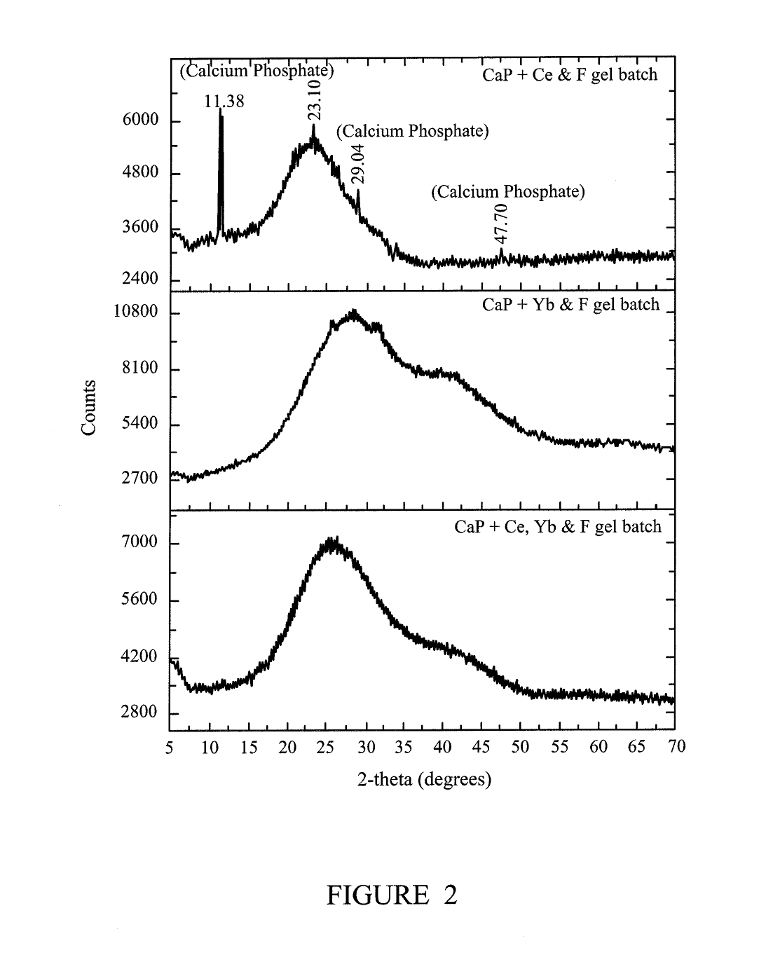

FIG. 2 is XRD spectra of the CaP gels;

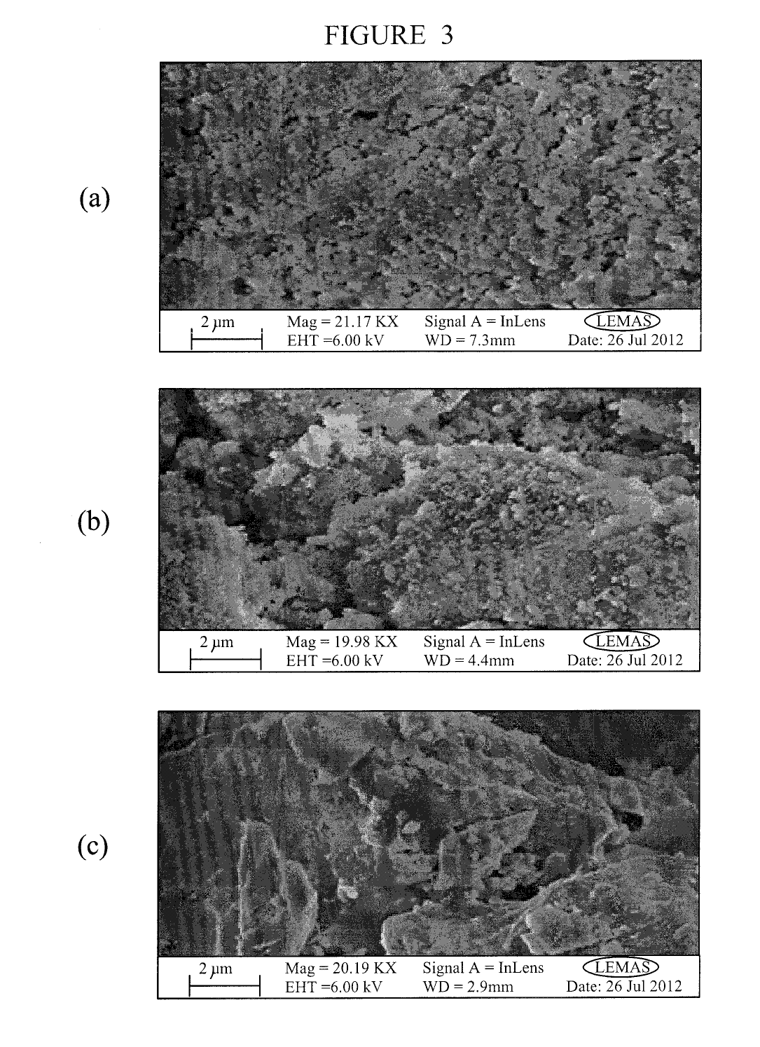

FIG. 3 is SEM images of the CaP gels during (a) particles agglomeration, (b) formation of platelet-like particles and (c) platelet-like particles;

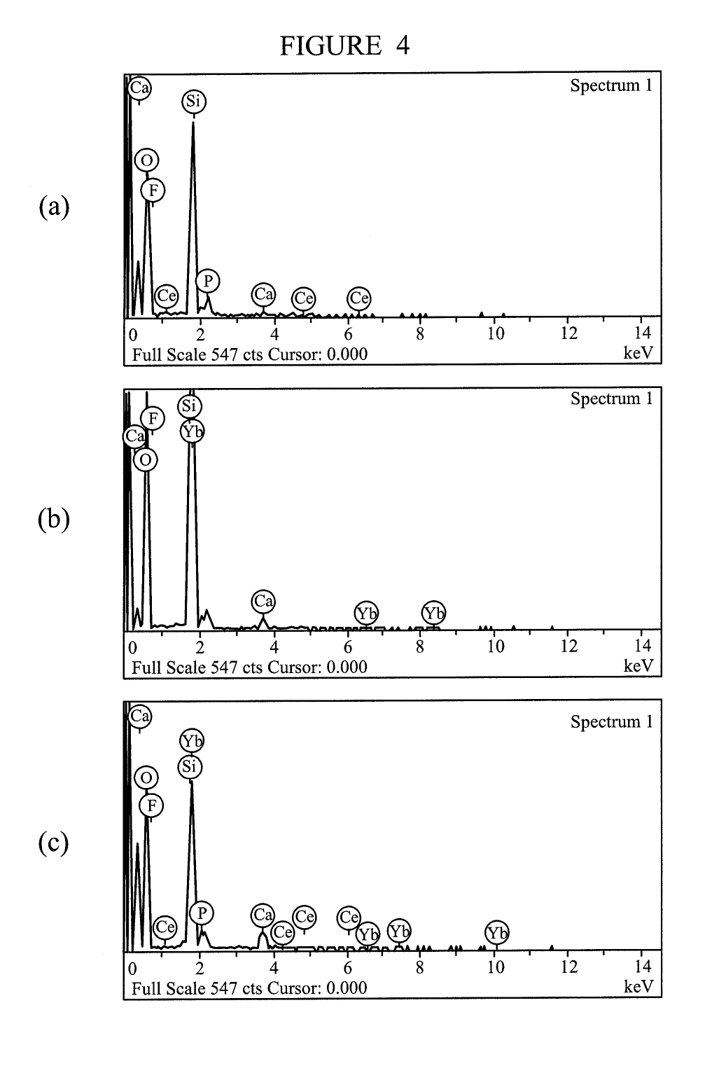

FIG. 4 is EDX spectra of the Ca-P gels doped with (a) Ce and F (b) Yb and F (c) Ce, Yb and F;

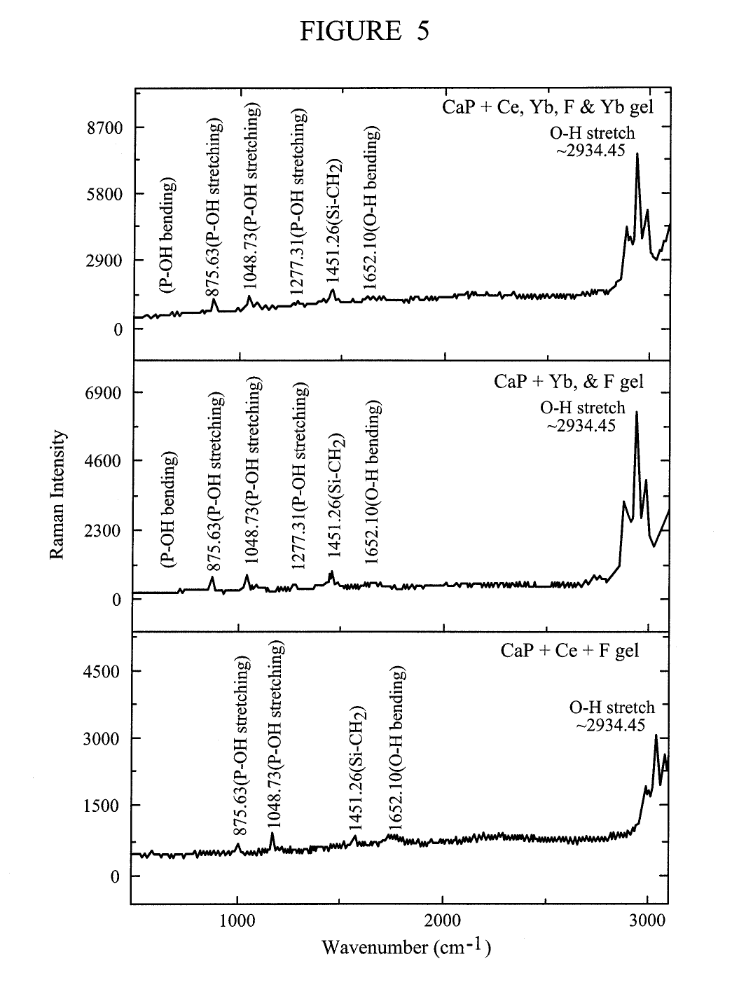

FIG. 5 is Raman spectra of the CaP gels with the various dopants;

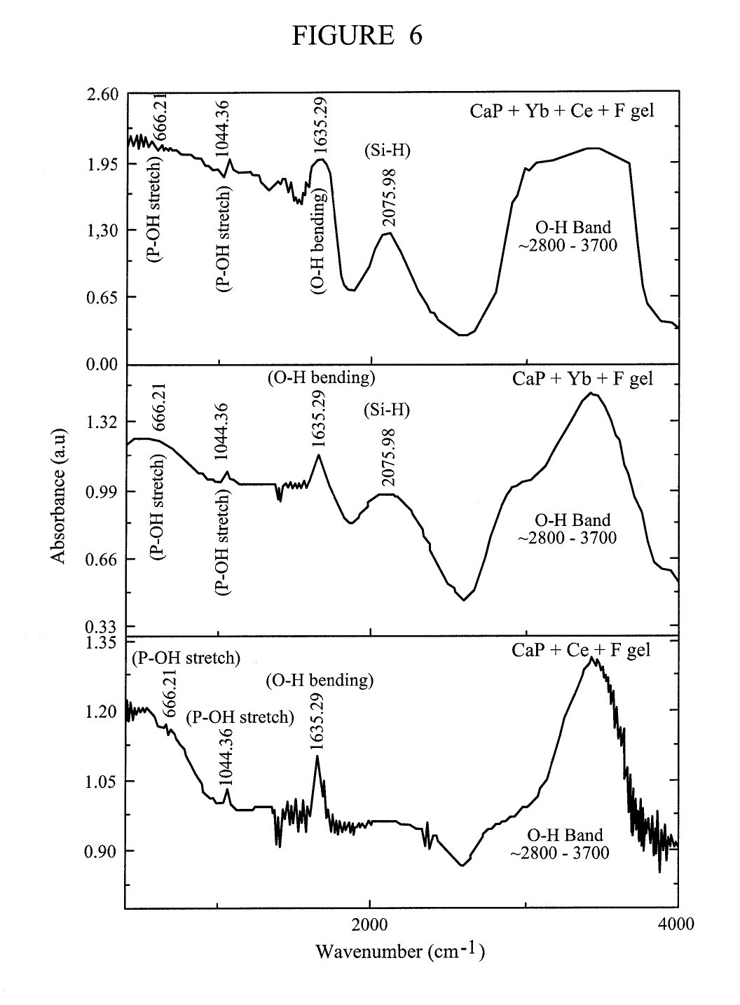

FIG. 6 is FTIR spectra of the CaP gels with the various dopants;

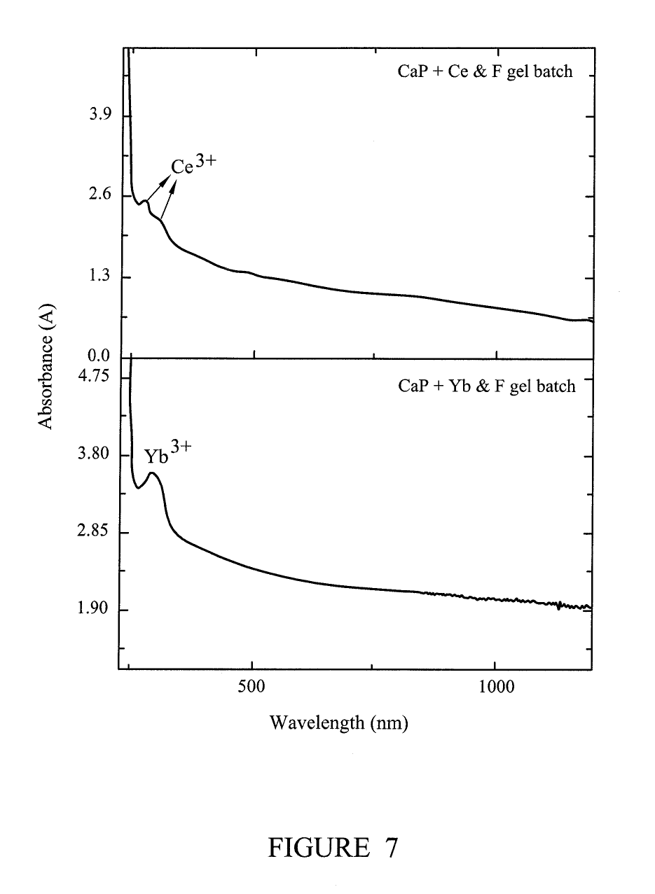

FIG. 7 is UV-V is spectra of the CaP gels with the various dopants;

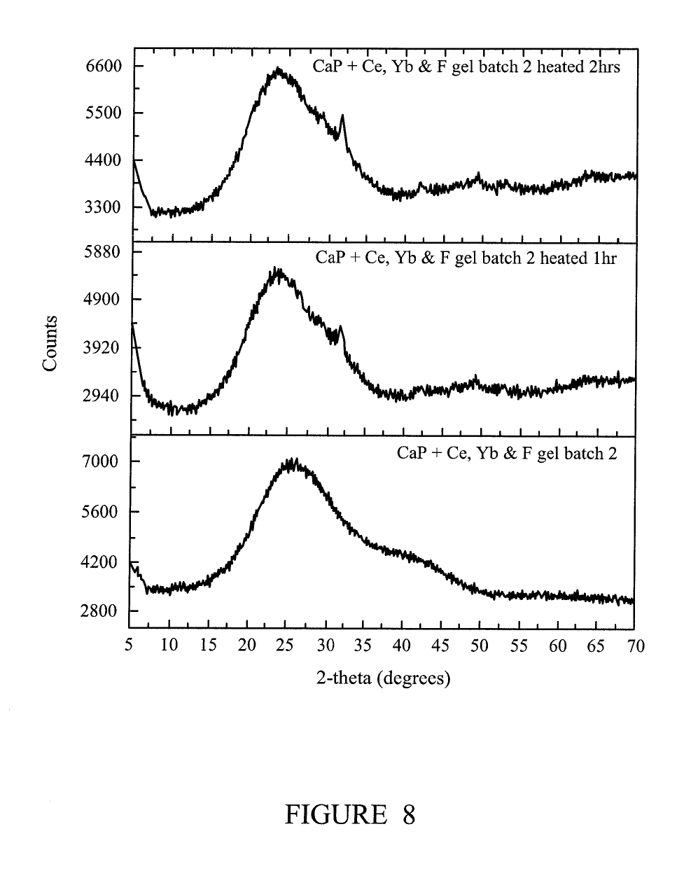

FIG. 8 is XRD analysis of CaP gel before and after heating at different times;

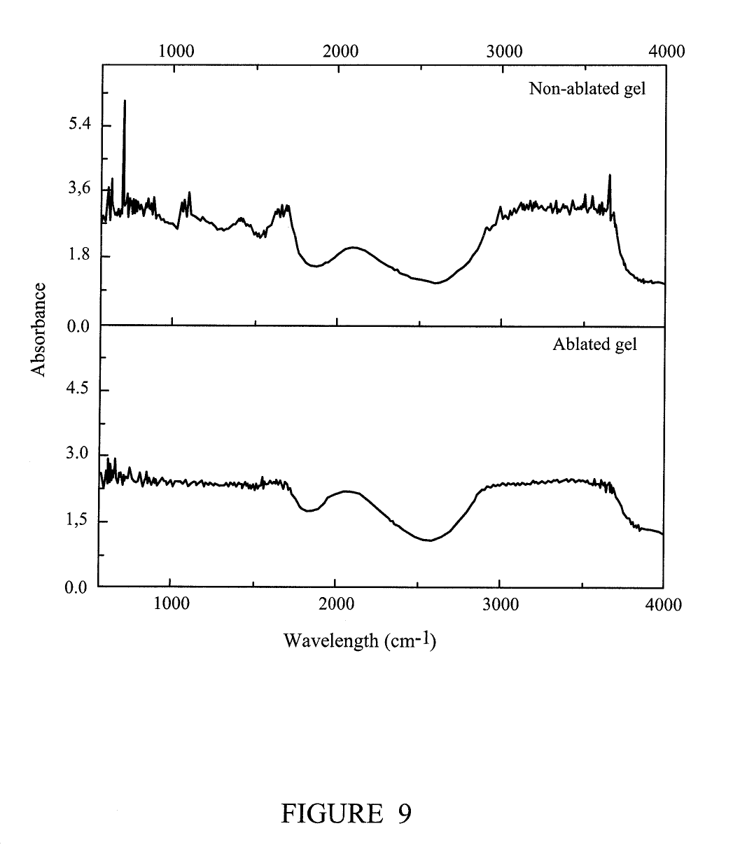

FIG. 9 is a comparison of the FTIR spectra of ablated and non-ablated CaP gels;

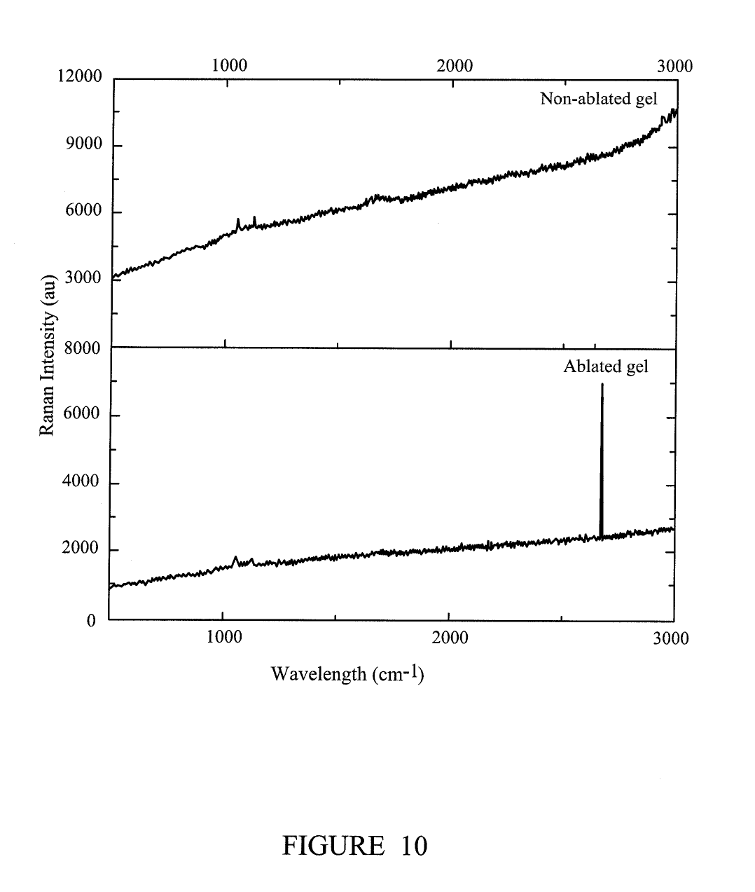

FIG. 10 is a comparison of the Raman spectra of ablated and non-ablated CaP gels;

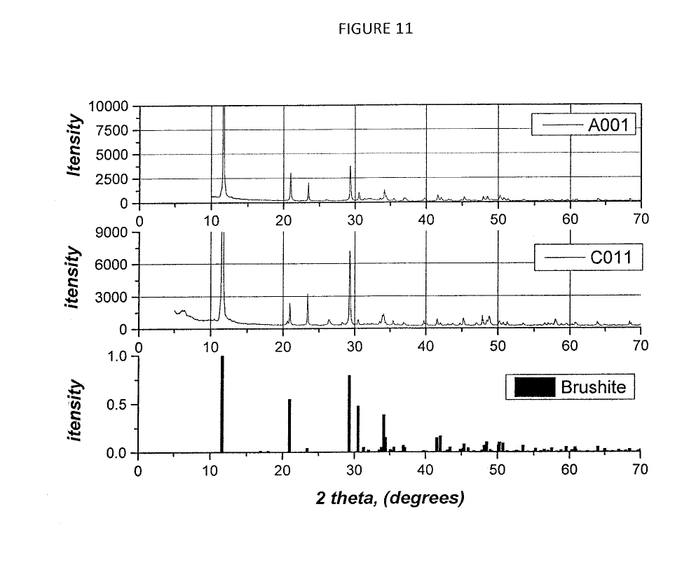

FIG. 11 is a comparison of A001 and C011 with the reference pattern of brushite;

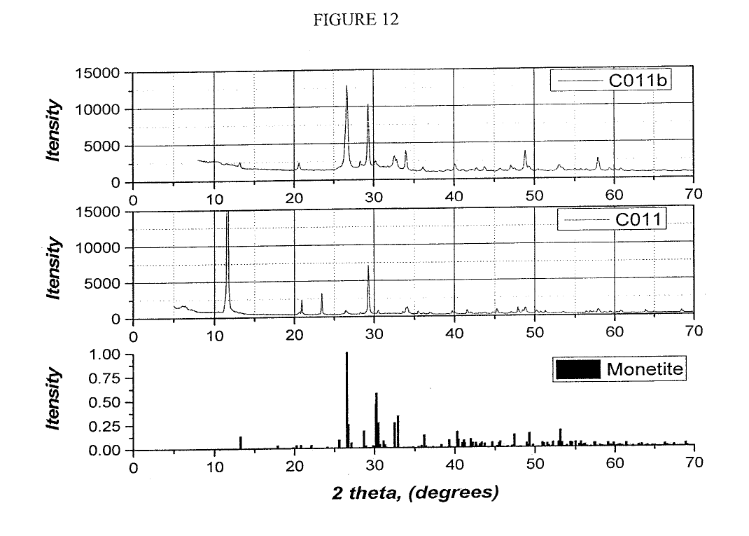

FIG. 12 is a comparison of C011 before and after (C011b) thermal treatment with the reference pattern of monetite;

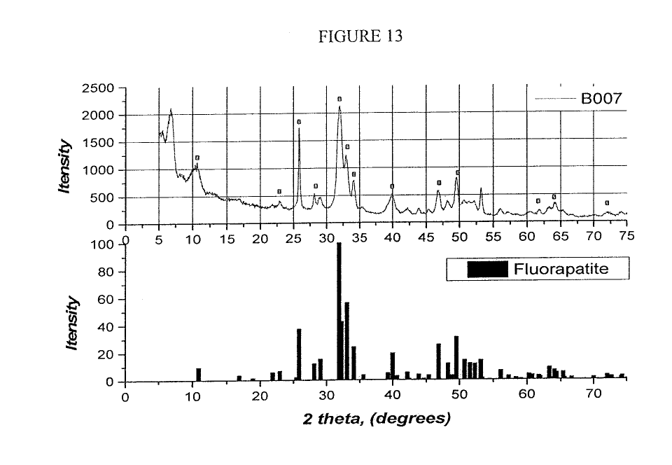

FIG. 13 is a comparison of B007 with the reference pattern of fluorapatite;

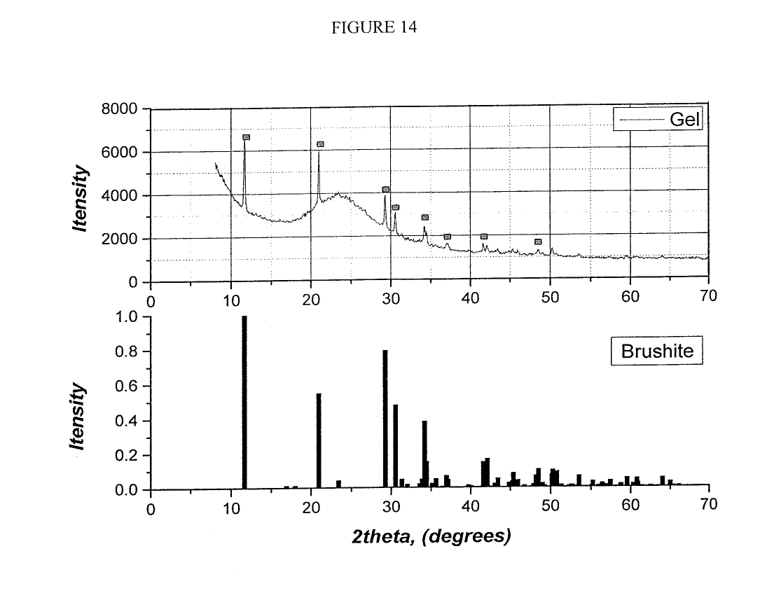

FIG. 14 is a comparison of CaP gel powder and the reference pattern of brushite;

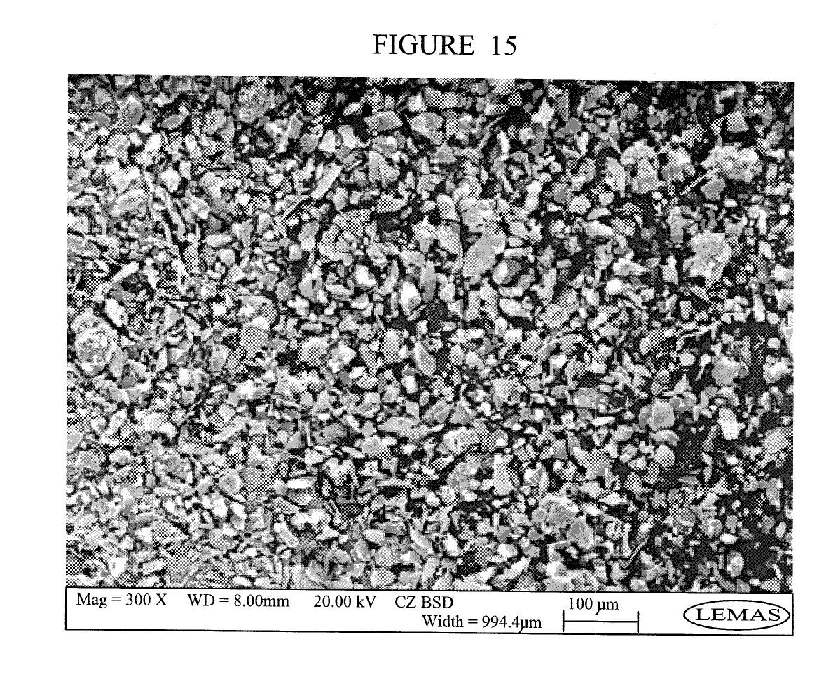

FIG. 15 shows SEM images of undoped brushite crystals (A001);

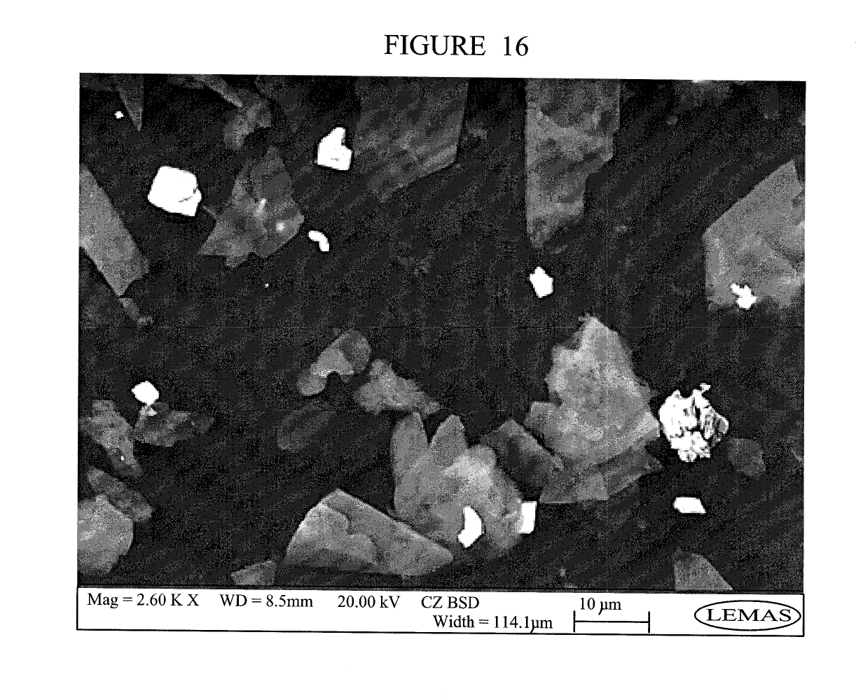

FIG. 16 shows SEM images of doped brushite crystals (C011);

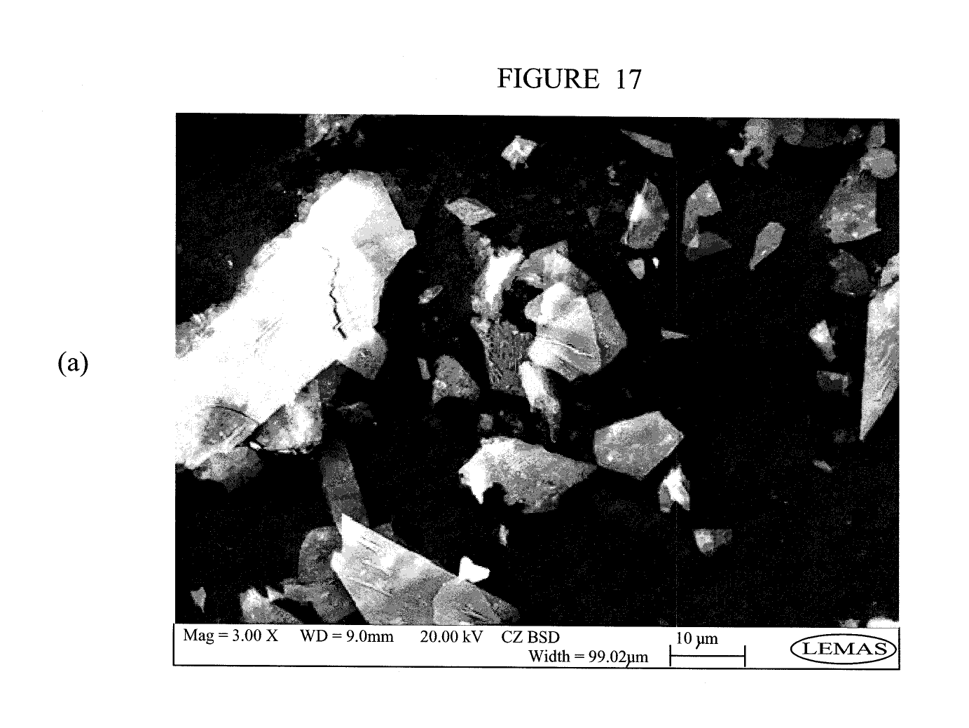

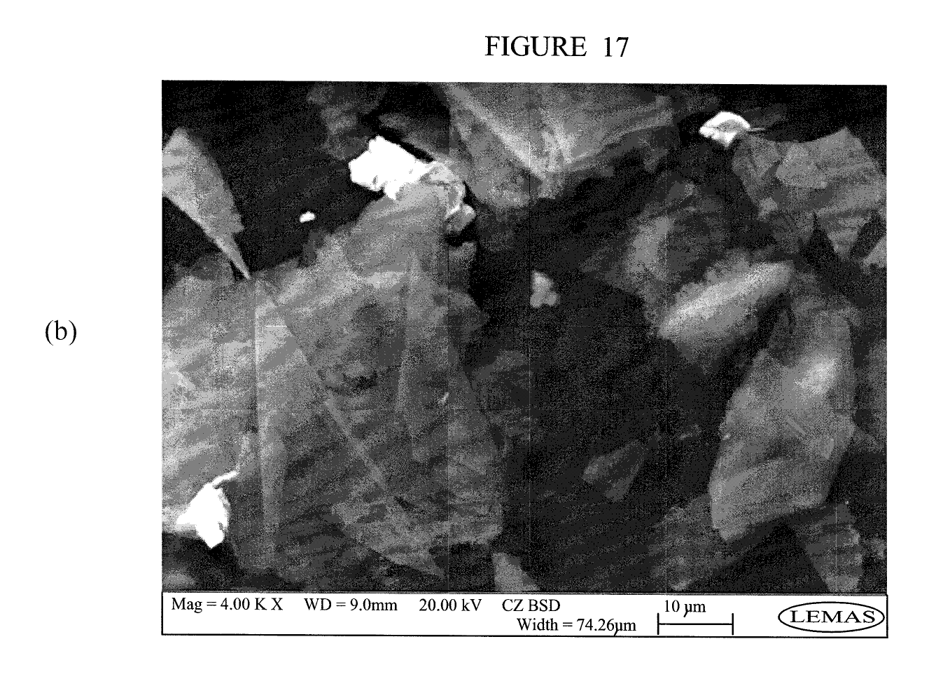

FIG. 17 shows SEM images of doped monetite crystals for C011b and b) doped monetite crystals for C012b;

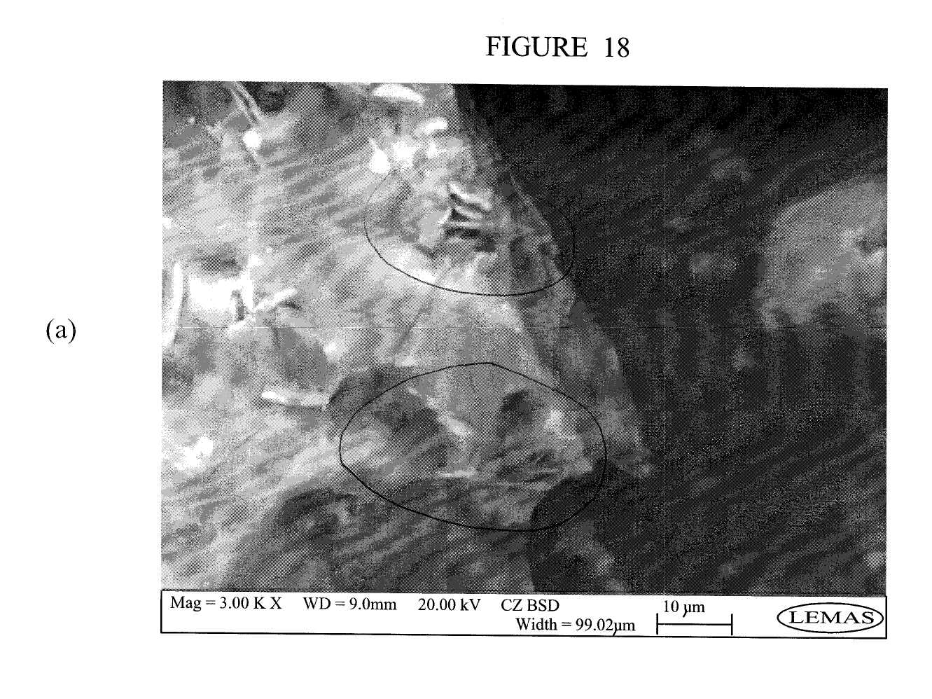

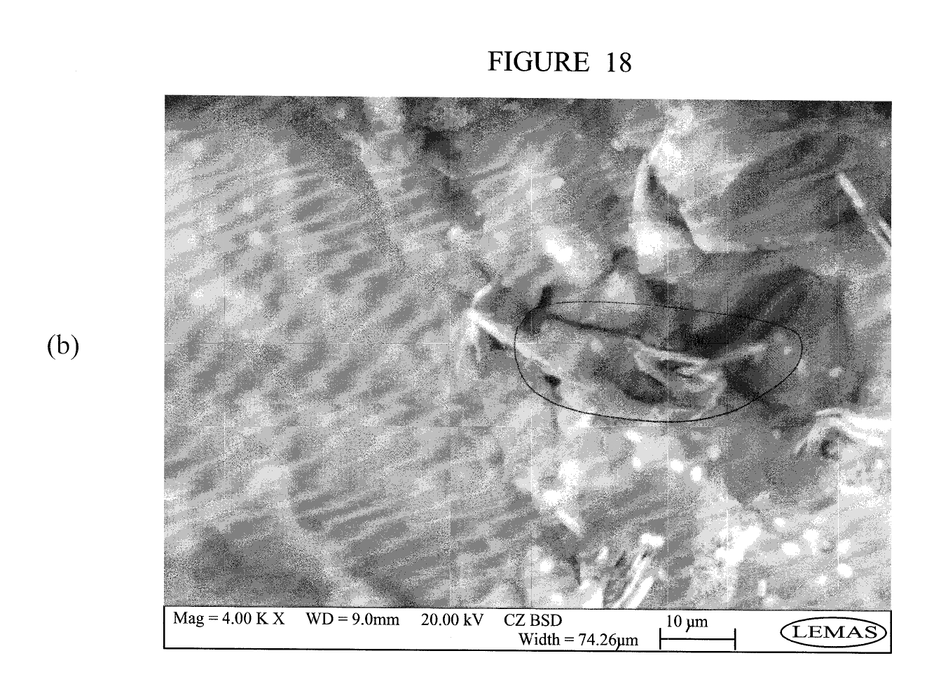

FIG. 18 shows SEM images of (a) CaP gel particles at 3 K X and (b) CaP gel particles at 4K X;

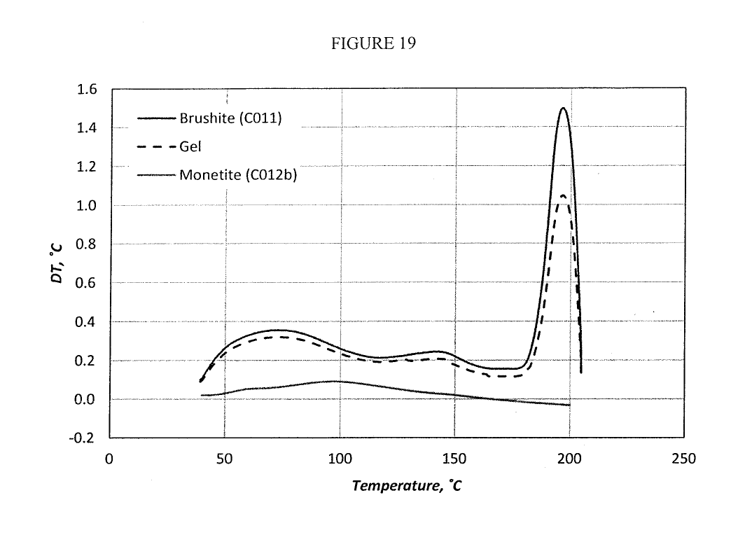

FIG. 19 is a thermal analysis for brushite, dried CaP gel and monetite;

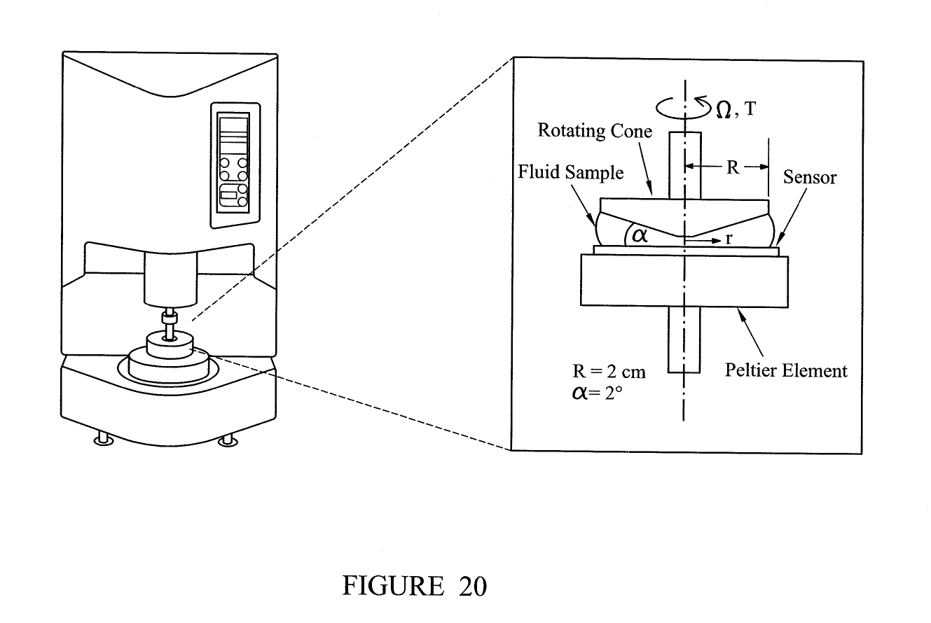

FIG. 20 is a Bohlin Gemini II rheometer and a cone-plate geometry;

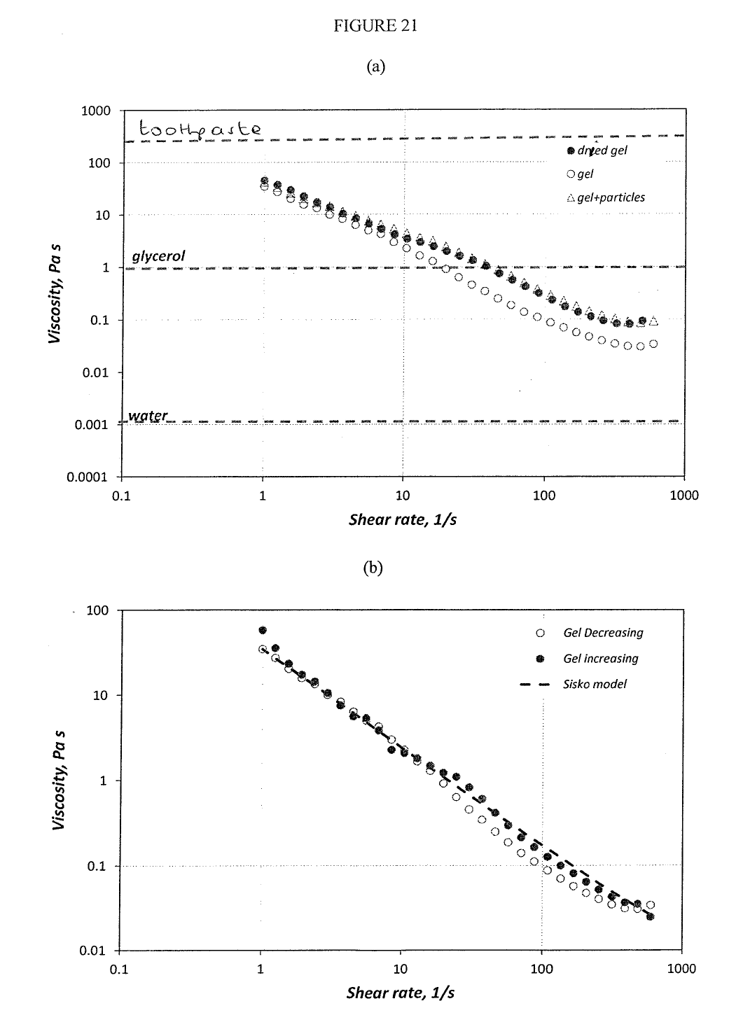

FIG. 21 is a) viscosity results for the three samples tested and compared with conventional fluids and b) fitting of the Sisko model to the viscosity data;

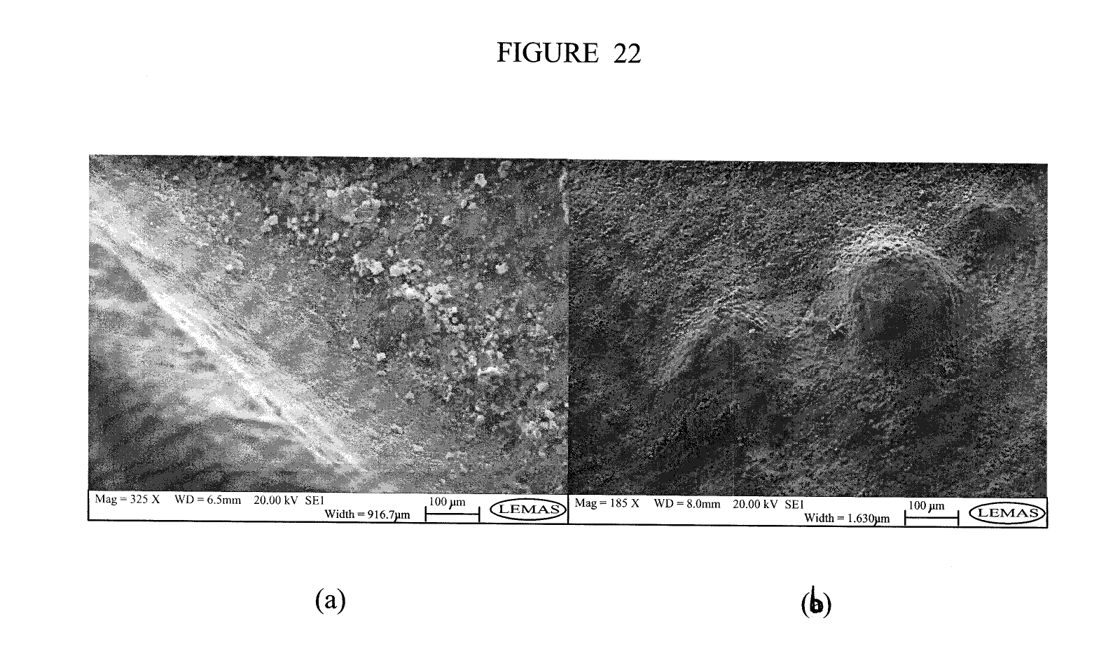

FIG. 22a (left) shows bio-mineral bonded with bovine incisors which were acid-eroded. In

FIG. 22b (right) the small pillars are the areas where laser irradiation was performed after which the bovine incisors were tested for 3 weeks brushing trials in an oral pH environment using 200 g brush load 4 times a day;

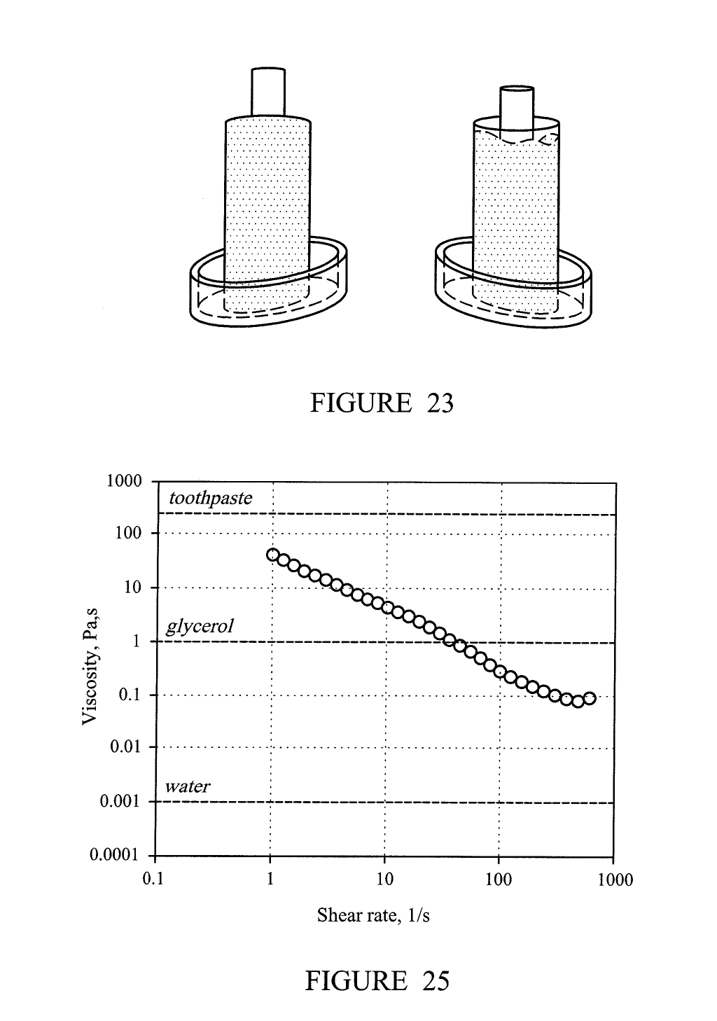

FIG. 23 shows cast hydrogel materials for making hollow bone shapes for investigation of osteoinduction, conduction and osseointegration;

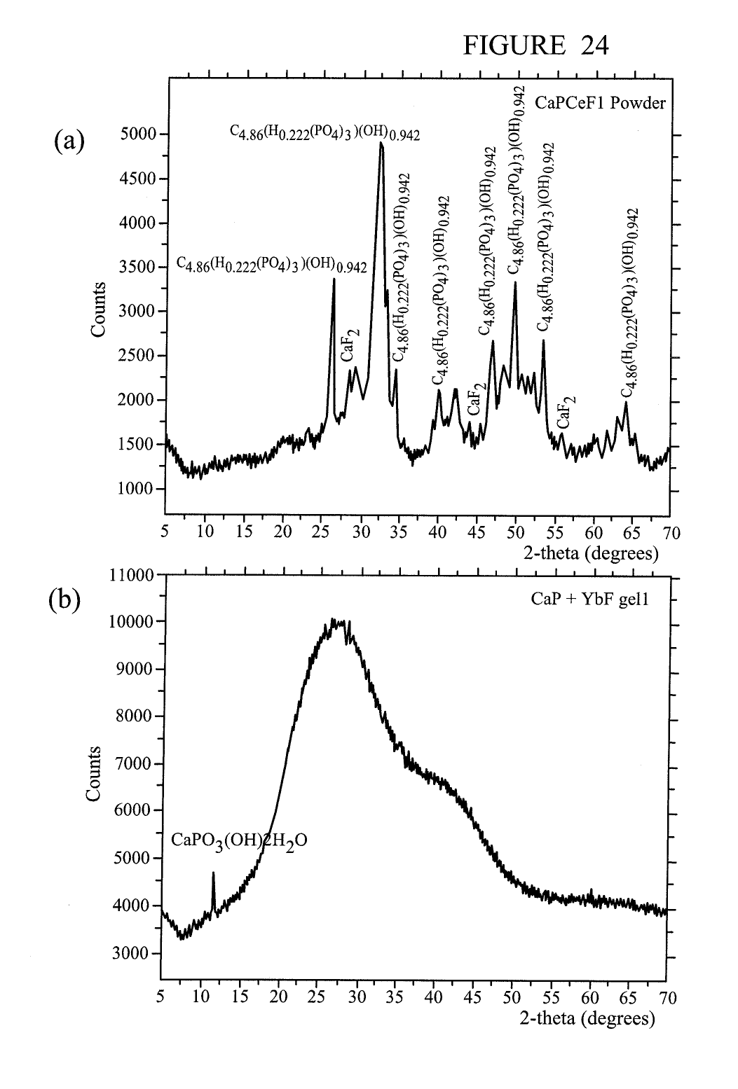

FIG. 24a (left) is a X-ray powder diffraction pattern of laser crystallised gel powder which was derived from cast materials shown in FIG. 23. Before laser crystallisation the hydrogel is largely amorphous as shown in FIG. 24b (right);

FIG. 25 shows viscosity measurement of a mixture of chitosan and t-orthosilicate after mixing with brushite crystals (10:1 ratio) at 25° C.;

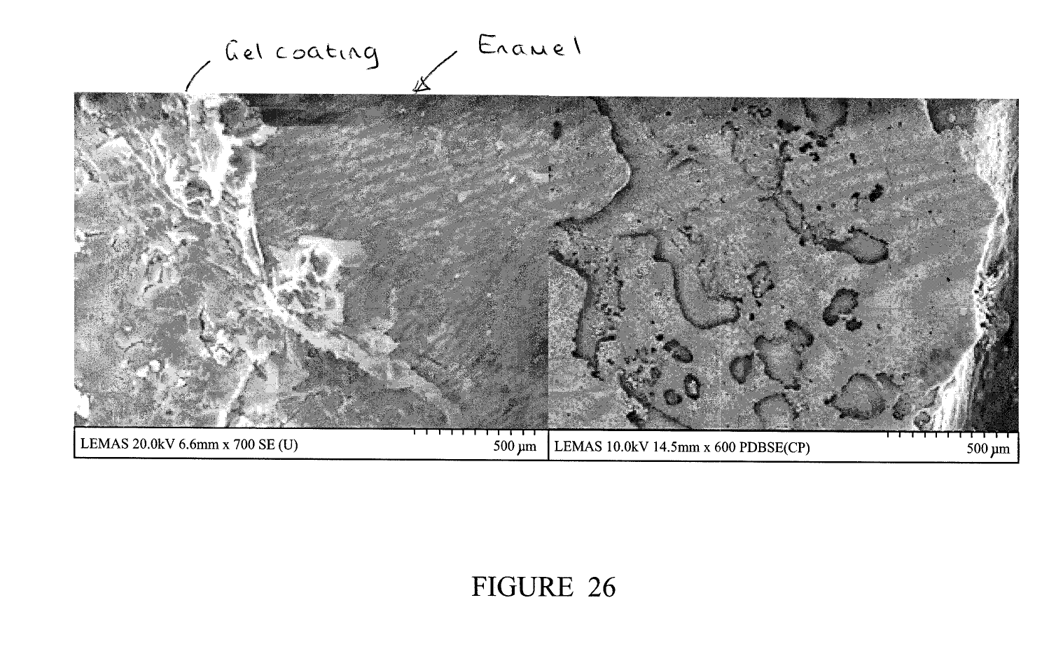

FIG. 26 shows enamel samples coated with the hydrogel formulation of Example 5 a) before laser irradiation and b) after laser irradiation (fs-p 1 GHz repetition rate, 30 pm spot size and 0.4 W average power); and

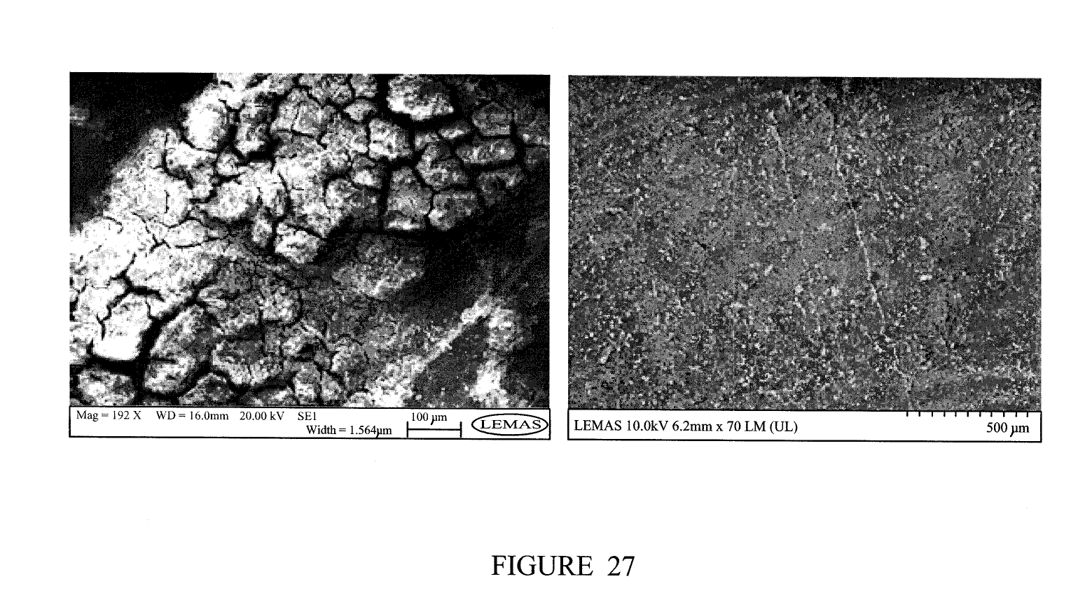

FIG. 27 is a comparison of a coating of a) a t-orthosilicate hydrogel formulation with b) a chitosan and t-orthosilicate hydrogel formulation.

### EXAMPLE 1

Synthesis

Different procedures were used to synthesise two batches of CaP gel.

For batch 1, 37.5 mL of a 0.1M (NH₄)₂HPO₄ solution was added dropwisely to 75 mL of 0.1M Ca(NO₃)₂.4H₂O solution with continuous stirring. Then 3.75 mL each of 0.1M of Yb(NO₃)₃.5H₂O solution and NH₄F solution were added dropwisely under continuous stirring. The mixture was left to stir for about 24 hours then 30 mL of tetraethylorthosilicate was added with stirring. The solution was stirred for about 2 hours and then left to form a CaP gel at about 25° C.

For batch 2, 10 ml of tetraorthosilicate was added dropwisely to 25 mL of 0.1M Ca(NO₃)₂.4H₂O solution with continuous stirring. 12.5 mL of 0.1M (NH₄)₂HPO₄ was also added, followed by 1.25 mL each of 0.1M of Yb(NO₃)₃.5H₂O and NH₄F solution with continuous stirring. The mixture was stirred for about 24 hours and then left to form a CaP gel at about 25° C.

The procedure used to prepare batch 2 was also used to prepare a CaP gel doped with cerium and fluorine and a CaP gel doped with cerium, ytterbium and fluorine.

Characterization

The CaP gels were subjected to structural, spectroscopic and thermal analysis.

X-ray diffraction patterns of the dried CaP gels and powders were used to identify their crystal structures. Scanning Electron Microscopy was used to produce three-dimensional representations of the sample surface utilising its resolution abilities to give the distribution of the samples. The procedure involved initial coating of LEO stubs with gold, applying the samples on the coated LEO stubs, coating the sample with gold and then inserting into the SEM machine. Images were taken at different magnifications. Energy Dispersive X-ray was used to perform elemental analysis of a sample.

UV-V is Spectroscopy was used to measure the absorbance or transmittance of UV light through a sample using a spectrometer. This involved the preparation of a sample suspension by diluting and thoroughly mixing with distilled water at a ratio of 1:1 in an ultrasonic bath for approximately 5 minutes and then transfer into a cuvette in the sample holder. Absorption spectra due to the different energy levels were observed and used to predict/identify the chemical ions present in the sample. Raman Spectroscopy was used to obtain the identity and crystal orientation of a sample by analysing the different energy frequencies (eg vibrational or rotational). FTIR Spectroscopy was also used for identification and composition studies of the samples by giving the absorption and emission spectra. The samples were placed between two KBr windows which were run as the background where it would be measured at the MIR and NIR.

The CaP gels were heated to a given temperature and subjected to phase analysis by X-Ray diffraction. Ablation studies were performed on the samples by laser irradiation at very low energy (less than 10 μm) and a wavelength of 800 nm. Femtosecond lasers were used to excite the CaP gels at very short time intervals (1 min) for 5 minutes and changes in weight were measured to determine changes in density associated with water loss and laser excitation. The ablated CaP gels were then analysed by Raman and FTIR and the results compared with those from non-ablated CaP gels.

Results and Discussion

(1) Synthesis

From FIG. 1, it can be seen that an attempt to synthesize CaP gels doped with ytterbium and fluorine in batch 1 yielded products which were initially in three phases. The upper phase consisted mostly of water. The lower phase consisted mostly of tetraethylorthosilicate. The powder-like particles in the precipitate beneath consisted mostly of the Ca, P, Ce and F phases. After a while, the mixture started to thicken to form a CaP gel as hydrolysis and diffusion began to occur. Water in the upper phase was absorbed and the powder phase was dispersed from the region of high concentration to the region of lower concentration.

The product in batch 2 was fairly homogenous and thick within about 24 hours. By adding tetraethylorthosilicate to Ca(NO₃)₂.4H₂O first, the reaction had been enhanced. The increased stirring time of the solution including the tetraethylorthosilicate helped to speed up the diffusion process and explains the more rapid formation of CaP gel in batch 2. Since gelation occurs by hydrolysis via diffusion, the lower volume of batch 2 offers an advantage over that of batch 1.

(2) X-Ray Diffraction

Handsvolt method was employed for the identification of the XRD peaks and with the use of Xpert hands plus software, the different peaks were identified. The XRD spectra shown in FIG. 2 are typical of an amorphous material and confirm the presence of an amorphous gel. A few peaks were detected at points 11.38, 23.10, 29.04 and 47.70 (4, 11) corresponding to brushite. In summary, an amorphous CaP gel having a brushite nature was formed.

(3) SEM Analysis

The CaP gel showed the platelet-like structural packing which is characteristic of hydroxyapatites (see FIG. 3). Phase analysis data was employed to determine the predominant phases in the sample. Thus from FIG. 2 it was shown that the CaP gels contained mostly brushite.

(4) Energy Dispersive X-ray

FIG. 4 shows the EDX spectra for portions of the samples and indicates the different elements which are present in each mapped area. Calcium has the highest concentration followed by silica then oxygen and phosphate. Cerium, ytterbium and fluorine were found in very minute quantities. The equipment was unable to detect the very low energy of hydrogen.

(5) Raman Spectroscopy

After subtraction of the KBr windows from the peaks, the Raman spectra was obtained and analysed (FIG. 5). The peak at 428.78 was assigned to the P—OH bending vibrations as it corresponds to the V₂ bending vibrations of the PO₄²⁻ ion. The peak at 586.91 was also assigned to P—OH bending as it corresponds to the V₄ vibrational mode from degenerate bending vibrations. Peaks at 875.63, 961.91, 1048.73 and 1277.31 were assigned to P—OH stretching as they correspond to symmetric Vi vibrational stretching. The peak at 1652.10 was assigned to O—H bending which results from the water overtone and the peak at ˜2934.45 was assigned to O—H stretching from water.

(6) FTIR Spectroscopy

Analysing the FTIR data in FIG. 6, P—OH stretching vibrations resulting from the symmetric V₁ and asymmetric V₃ modes were assigned to the peaks observed at 666.21 and 1044.36 confirming the presence of calcium phosphate. The P—OH vibrations at 666.21 were assigned specifically to vibrations from phosphates of apatite nature. The peak at 1635.29 was assigned to the O—H bending mode of water. The peak at 2075.96 was assigned to the Si—H vibration from the silicate gelling material. At ˜2800-3700, there was a very broad peak assigned to the hydrogen bonded O—H stretching vibration of water and HAp.

Thus from the complementary Raman and FTIR results, it was seen that similar vibrational peaks were observed at similar wavelengths for O—H and P—OH bonds. The O—H vibrations from water were consistent with the formation of hydrogel and the P—OH vibrations were consistent with the formation of apatite.

(7) UV-Vis Spectroscopy

In the CaP gels containing cerium, a peak was observed at around 271.16 alongside a smaller peak at 301.77 which is consistent with the literature value of about 300 nm (Zinkstok et al. Journal of Physics. B, Atomic, molecular and optical physics: an Institute of Physics Journal., 2002, 35, pp. 2693-2702). For the ytterbium doped CaP gels, peaks were observed at a wavelength of 296.50 which is quite different from most literature reviews (˜980 nm) but consistent with Zinkstok et al where the isotope shift for the three UV-transitions were measured and different wavelengths were identified for different isotopes and where the closest wavelength was at 267.28 nm.

(8) Crystallization of CaP Gels

From the XRD spectra in FIG. 8, the absence of any sharp peaks showed that prior to heating, the CaP gel was amorphous. However after heating for 1 hour, several broad and small peaks showed that there was a transformation from amorphous to crystalline phase. On further heating and analysis, the peaks got sharper and thinner indicating increased crystallinity.

(9) Characterisation of Laser Ablated CaP Gels

The weight of the CaP gel was recorded after each interval and is presented in Table 1. At the onset, weight loss was high possibly due to the presence of large amounts of loose minerals. However the rate of weight loss became lower over time suggesting that few loose minerals remained.

TABLE 1
Weight of ablated CaP gel sample with time

| Time | Sample weight |
|---|---|
| (secs) | (g) |
| 0 | 43.268 |
| 60 | 43.192 |
| 120 | 43.173 |
| 180 | 43.161 |
| 240 | 43.147 |
| 300 | 43.131 |

FIG. 9 shows only slightly differences in the FTIR spectra of the ablated and non-ablated CaP gels which suggests few changes occurred. Firstly there is the presence of noise or absence of P—OH peaks before 1800 cm⁻¹ rather than sharp peaks. Secondly there is a hypochromic shift at the broad peak identified to be from water at ˜2800-3700 resulting from loss of water from the CaP gel. These changes confirm the loss of water and associated structural changes in the CaP gel.

FIG. 10 compares the Raman spectra of the ablated CaP gels with the non-ablated CaP gels. Similar peaks were observed at 1048.73 for phosphate bond vibration. The water peak which was observed for the non-ablated CaP gels at ˜2934.45 was missing for the ablated CaP gels confirming a significant loss of water. Most significantly, there was a very sharp peak at ˜2605.05 resulting from HPO₄²⁻ absorption. It was apparent that laser ablation led to a significant loss of water and densification.

Comparing the Raman results with the FTIR, the absence of P—OH peaks before 1800 cm⁻¹ in FIG. 9 cannot be attributed to loss or damage of phosphate bonds because the P—OH vibration peak was present at 1048.73 and 1277.31 in the Raman spectra (FIG. 10). Both techniques show that water has been lost from the material with the thin very sharp HPO₄²⁻ absorption peak (FIG. 10) indicating crystallization.

Conclusions

For the synthesis of doped CaP gels, nitrate solutions of Ce, Yb and F were incorporated into stock solutions of Ca(NO₃)₂.4H₂O, (NH₄)₂HPO₄ and TEOS. Spectroscopic analysis showed the presence of P—OH and O—H bonds from HAp and hydrogels. Phase analysis showed a predominance of brushite. SEM analysis revealed a platelet-like structure. Laser ablation of the CaP gels resulted in weight loss, structural modification and densification due to loss of water.

### EXAMPLE 2

The aim of this Example was to develop a suitable material which after laser sintering would effectively protect tooth enamel from erosion. Four materials were synthesised as follows:

Brushite-containing material.

Monetite-containing material.

Fluorapatite-containing material.

CaP gel particles.

Material Synthesis

Brushite

The synthesis of brushite can be divided into five steps:

(a) Preparation of the 1M stock solutions: For the first solution, 47.230 g of Ca(NO₃)₂.4H₂O were placed in a volumetric flask. Water was added until the total volume was 200 ml. The mixture remained under constant stirring at room temperature for 10 minutes. For the second solution, 26.411 g of (NH₄)₃PO₄ were placed in a volumetric flask and water was added until the total volume was 200 ml. The mixture remained under constant stirring at room temperature for 10 minutes.

(b) Preparation of 0.1M solutions and preheating: For the preparation of the 0.1M calcium nitrate solution, 20 ml of the first stock solution were diluted with distilled water in a beaker (600 ml) until the volume reached 200 ml. After that the solution was placed on a heating plate and heated to 37° C. under continuous stirring. For the preparation of the 0.1M ammonium phosphate solution, 20 ml of the second stock solution were diluted with 180 ml of distilled water in a beaker until the volume reached 200 ml. The mixture was placed in a burette of 200 ml. During the synthesis there was continuous monitoring of the temperature with a K-type thermocouple.

(c) Mixing of the raw materials: When the calcium nitrate solution reached 37° C., the ammonium phosphate solution (0.1M) was added dropwisely through a burette. During the addition of the (NH₄)₃PO₄ a slight decrease of the temperature was noticed (35° C.). After mixing, the pH was measured. Normally it should be between 5.3 and 5.6.

(d) Addition of dopants: Two different groups of dopants were tested. The first group consisted of 0.161 g Erbium oxide (Er₂O₃), 0.033 g Calcium Fluoride (CaF₂) and 0.053 g Aluminium phosphate (AlPO₄). The second group consisted of 0.1850 g Erbium Nitrate [Er(NO₃)₃.5H₂O], 0.033 g Calcium Fluoride (CaF₂) and 0.1660 g Aluminium Nitrate [Al(NO₃)₃.9H₂O]. In both cases, the dopants in crystalline form were added to the (NH₄)₃PO₄ and Ca(NO₃)₂.4H₂O mixture and stirred at 37° C. for 1 hour.

(e) Precipitation, filtration and drying: The mixture was allowed to settle for 1 hour for precipitation and then the brushite crystals were collected on a filter paper (Whatman grade 44). The crystals were dried for 24 h to 80° C.

During the synthesis of brushite, several parameters were altered in order to investigate how they affected the final product. It was found that parameters such as the Ca:P ratio and the mixing time (step d) had no effect on the brushite crystals. On the other hand more significant seem to be the mixing temperature, the pH of the mixture and mass of the added dopants.

Monetite

Brushite crystals were placed in an oven for 72 hours at 200° C. The time of 72 h was chosen to ensure complete transformation of the brushite to monetite but this could probably be achieved in a shorter time.

Fluorapatite

For the synthesis of fluorapatite, steps a, b, c and e were the same but the dopants which were added during step d were different. The production of fluorapatite was achieved by replacing CaF₂ with NH₄F. The addition of NH₄F decreased the pH from 5.5 to 4.6 while the solubility of NH₄F was higher than CaF₂ and consequently more F⁻ions were available to react with the CaP crystals.

CaP Gel

The method for the preparation of the CaP gels can be divided into the following four steps:

Step 1: 0.033 g of NH₄F was added to 100 ml of (NH₄)₂HPO₄solution (0.1M) and the mixture was stirred for 5 minutes.

Step 2: 100 ml of Ca(NO₃)₂.4H₂O solution (0.1M) was heated to 37° C. After that the mixture of (NH₄)₂HPO₄and NH₄F was added dropwisely under continuous stirring. At the same time, 0.185 g ErNO₃ and 0.166 g AlNO₃ were added in powder form. The mixture was stirred for 10 minutes.

Step 3: 50 ml of tetraethylorthosilicate was added instantly to 200 ml of the mixture (ratio of 1:4). The mixture was stirred for about 1 hour to 37° C.

Step 4: The mixture was stirred for 72 hours at room temperature (˜25° C.) to form a CaP gel. If the mixture is not continuously stirred, three phases are formed. At the bottom are the precipitated CaP particles. In the middle is the water phase. At the top is the unreacted orthosilicate which is less dense than water (0.93 g/ml). Continuous stirring promotes the homogeneity of the CaP gel.

Another important observation was that gelation was not complete for the undoped mixture. It may be assumed that the reason for that is the final pH. With the addition of the NH₄F the pH is about 4.5 while for the case of the undoped CaP gel the pH is about 5.3. Consequently for the production of undoped CaP gel the pH must be adjusted with the addition of an acid.

These procedures have been used to prepare many samples and the most representative ones are presented in Table 1. Sample A001 is undoped brushite, A001b is undoped monetite, B006 and B007 are fluorapatites, C011 and C012 are brushite doped with different minerals and C011b and C012b are the respective monetites.

TABLE 1
Representative cases of the CaP minerals produced

| Code | Temperature, ° C. | pH | Dopants | Thermal treatment |
|---|---|---|---|---|
| A001 | 37 | 5.5 | — | no |
| A001b | | | — | 200° C. for 72 h |
| B006 | 37 | 4.7 | Er(NO₃)₃•5H₂O | no |
| | | | Al(NO₃)₃•9H₂O | |
| | | | NH₄F | |
| B007 | 37 | 4.7 | NH₄F | no |
| C011 | 37 | 5.3 | Er₂O₃ | no |
| | | | AlPO₄ | |
| | | | CaF₂ | |
| C011b | 37 | 5.3 | Er₂O₃ | 200° C. for 72 h |
| | | | AlPO₄ | |
| | | | CaF₂ | |
| C012 | 37 | 5.5 | Er(NO₃)₃•5H₂O | no |
| | | | Al(NO₃)₃•9H₂O | |
| | | | CaF₂ | |
| C012b | 37 | 5.5 | Er(NO₃)₃•5H₂O | 200° C. for 72 h |
| | | | Al(NO₃)₃•9H₂O | |
| | | | CaF₂ | |

XRD Characterisation

The synthesized powders were analysed using the X-Ray powder diffraction technique on a D8 discover, Brucker using monochromatic CuKa 0.154098 nm radiation. For the characterisation of the powders, the step size was 0.062° and the scanning range was 5° to 70° over a period of approximately 25 minutes.

In FIG. 11 the patterns for an undoped (A001) and a doped (C011) sample are presented in comparison with the reference pattern of brushite (Reference code 01-074-6549). In both cases it is clear that brushite is the dominant phase. For the undoped sample, all of the characteristic peaks match with the reference pattern (for 2 theta 11.58, 21.09, 30, 30.6, 34.2). For the doped sample, the characteristic peaks of brushite are present with peaks attributable to the dopants (ie the strong peak at 29.2 and the peaks at 48.9 correspond to erbium oxide pattern). The relative intensity of the peaks is quite different from the reference pattern as the peak for 11.6 degrees is much higher than the others. This is due to the texture of the brushite crystals (ie distribution of crystallographic orientations of a polycrystalline sample).

In FIG. 12 a comparison is made between the brushite crystals (C011), the sample after the thermal treatment (C011b) and the reference pattern of monetite (Reference code 00-009-0080). The transformation into monetite is clear. In sample C011b the characteristic peaks of brushite are absent but the characteristic peaks of monetite are present (2 theta 13.24, 26.58, 28.73, 30.30). The phenomenon of texture is present for monetite as the peak at 26.58 degrees is relatively high in comparison with the reference pattern.

FIG. 13 is the pattern of sample 8007 and the reference pattern of fluorapatite (Reference code 04-007-2771). All the characteristic peaks are recognisable (2 theta 10.61, 23.08, 25.88, 28.1, 29.09, 32.15, 33.01, 34.14, 40.04, 46.70) while the phenomenon of texture is not present.

For the characterisation of the CaP gel with XRD, a small amount (20 ml ) is left in a beaker at room temperature for 24 hours to dry into a powder form. In FIG. 14 the XRD pattern of the CaP gel is compared with the reference pattern of brushite. The presence of brushite crystals in the CaP gel is clear while at the same time there is a broad peak (2 theta 20 to 30) which indicates the presence of an amorphous phase (gelated orthosilicate). The presence of this amorphous phase ensures the random alignment of the brushite crystals and no texture was therefore observed.

SEM Analysis

Scanning Electron Microscopy was used to investigate the shape and the size of the crystals. In FIG. 15 images of the undoped brushite powder (A001) are presented. In this case the crystals have the shape of a flake with a length of 5-80 μm and a width of 3-10 μm. The same characteristics were observed for the doped samples (FIG. 16) but unreacted dopants were also found (white particles correspond to erbium oxide). The same shape remains after the dehydration of brushite and its transformation to monetite. The only difference between brushite and monetite is that in the second case rough areas can be found on the surface of the crystals (see FIG. 17a and FIG. 17b). These rough areas can be attributed to the formation of faults. The appearance of stacking faults is very common during phase transition and generally during the heating and cooling of crystals. The flake shape of brushite and monetite favours the alignment of the particles and can explain the high peaks due to texture which have been observed during XRD analysis.

In FIG. 18 the dried CaP gel particles can be observed. The dominant phase is the particles of orthosilicate polymer but with recognisable flake-like crystals of brushite trapped in it (see FIG. 18a and FIG. 18b).

Thermal Analysis

A Simultaneous Thermal analyser (PerkinElmer, STA 8000) was used to investigate the reactions and the phase changes which take place during heating. Experiments were conducted in the range 40 to 200° C. at a heating rate of 10° C. per minute. In FIG. 19 the curves for a brushite sample (A001), a dried CaP gel and monetite (C011b) are presented. In the brushite sample and in the CaP gel sample there is indicated a phase transformation at 198° C. This was expected as it indicates the removal of water molecules from the crystals and the transformation of brushite to monetite. In the case of monetite no reactions or phase transformations have been found.

Viscosity Measurements

For the development of a suitable delivery system and the understanding of the mechanisms of coating on an enamel surface, rheology measurements of the CaP gels are very important. For that reason a Bohlin Gemini II rheometer was used on which the cone-plate geometry has been attached (see FIG. 20). Three samples were tested; a CaP gel, a mixture of CaP gel and brushite powder (10% w/v) and a dried CaP gel sample. The measurements were conducted at stable temperature of 25° C. and shear rates in the range 1-500 5⁻¹.

FIG. 21 presents viscosity results for the samples. It is clear that all of them are characterised by shear thinning behaviour (ie viscosity decreases with the increase of shear rate). As is to be expected the CaP gel has the lowest viscosity while the merely dried sample and the powder have almost the same viscosity curve. The samples were found to be much thicker than glycerol but thinner than toothpaste.

In order to proceed with Computational Fluid Dynamics (CFD) simulations a model which describes the viscosity of the fluids is needed. Several well-known viscosity models (Casson, Hershey Buckley, Sisko, Power law) have been tested by fitting to the experimental data. The most appropriate was found to be the Sisko model (eq. 1):

$$n = {n}_{∞} + {k ( \frac{1}{γ} )}^{n} \\ {n}_{∞} = apparent viscosity \\ γ = shear rate \\ k = viscosity constant \\ n = power law factor \qquad eq . 1$$

Conclusion

Four materials have been synthesised characterised respectively by the presence of brushite, monetite, fluorapatite and CaP gel. Synthesis is carried out at low temperature (37° C.) with solutions of Ca(NO₃)₂.4H₂O 0.1M and (NH₄)₃PO₄ 0.1M. Monetite can be produced by the dehydration of brushite (200° C. for 72 h) while fluoroapatite is formed when NFU is used as a dopant.

The brushite and the monetite crystals seem to be almost identical. Both are flakes with a length in the range 5-80 μm and width in the range 3-10 μm. Rheological measurements were conducted to determine the viscosity of the CaP gel. It was found that the samples follow a shear thinning behaviour which can be described using the Sisko model.

### EXAMPLE 3

Additional embodiments of the process of the invention are outlined briefly below.

General Methodology

Gel samples were prepared using a similar procedure which can be broken down into three steps, namely (a) preparation of the stock solution, (b) preparation of the solution and (c) gelation.

a) Preparation of the stock solution: each reagent is dissolved in double distilled water using a magnetic stirrer to give a stock solution with a concentration of 0.1M which was stored in a closed glass bottle.

b) Preparation of the solution: the dopants (cerium, fluorine and ytterbium) were added to the ammonium phosphate solution dropwisely under constant stirring after which the solution was left to stir for about 2 hours. In another beaker, the gelling material (tetraethylorthosilicate) was added dropwisely under continuous stirring to the calcium nitrate solution in the ratio 1:4 and left to stir for about 2 hours. Afterwards the solution mixture of ammonium phosphate and dopants was added dropwisely to the calcium nitrate and orthosilicate solution under continuous stirring.

c) Gelation: the prepared solutions were left under continuous stirring for about 24 hours for gelation to take place.

Preparation of Gel Samples

(1) Calcium Phosphate Doped with Cerium and Fluorine (batch 1):
7.5 mL of 0.1M Ce(NO₃)₂.4H₂O solution was added dropwisely to 75 mL of 0.1M (NH₄)₂HPO₄ solution with continuous stirring. 7.5 mL of 0.1M NH₄F solution was added in the same manner afterwards with continuous stirring.
The 90 mL phosphate solution with the dopants was then added dropwisely to a beaker containing 150 mL of calcium nitrate solution with continuous stirring.
The mixture was left to stir for about 24 hours.
62.5 mL of tetraethylorthosilicate was then added to the solution and left to stir for about 1 hr.
The mixture was left in a surrounding of about 25° C. to form a gel.

(2) Calcium Phosphate Doped with Cerium and Fluorine (batch 2):
3.75 mL of 0.1M Ce(NO₃)₃.6H₂O solution was added dropwisely to 37.5 mL 0.1M (NH₄)₂HPO₄ solution with continuous stirring. 3.75 mL of 0.1M NH₄F solution was added in the same manner afterwards with continuous stirring.
75.5 mL of 0.1M Ca(NO₃)₂.4H₂O solution was mixed with 62.5 mL of tetraethylorthosilicate (the ratio of tetraethylorthosilicate in the solution was increased as it was added to the calcium solution).
The 45 mL phosphate solution with the dopants was added dropwisely to a beaker containing the 138 mL of Ca(NO₃)₂.4H₂O solution with tetraethylorthosilicate with continuous stirring.
The mixture was left to stir for about 2 hours.
The mixture was left in a surrounding of about 25° C. to form a gel.

(3) Calcium Phosphate Doped with Cerium and Fluorine (batch 3):
3.75 mL of 0.1M Ce(NO₃)₃.6H₂O solution was mixed with 3.75 mL of 0.1M NH₄F solution and then added to 37.5 m1 of 0.1M (NH₄)₂HPO₄ dropwisely.
The 45 mL phosphate solution with the dopants was added dropwisely to a beaker containing 75.5 mL of 0.1M Ca(NO₃)₂.4H₂O solution and left to stir for about 1 hr.
30 mL of tetraethylorthosilicate was then added to the solution and left to stir for about 2 hrs.
The mixture was left in a surrounding of about 25° C. to form a gel.

(4) Calcium Phosphate Doped with Ytterbium and Fluorine (batch 1):
37.5 mL of 0.1M (NH₄)₂HPO₄ solution was added dropwisely to 75 mL of 0.1M Ca(NO3)₂.4H₂O solution with continuous stirring.
Then 3.75 mL each of 0.1M of Yb(NO₃)₃.5H₂O solution and NH₄F solution were added dropwisely under continuous stirring.
The mixture was left to stir for about 24 hrs.
30 mL of tetraethylorthosilicate was added with stirring. The solution was left to stir for about 2 hrs.
The mixture was left in a surrounding of about 25° C. to form a gel.

(5) Calcium Phosphate Doped with Ytterbium and Fluorine (batch 2):
10 mL of tetraorthosilicate was added dropwisely to 25 mL of 0.1M Ca(NO₃)₂.4H₂O solution with continuous stirring.
12.5 mL of 0.1M (NH₄)₂HPO₄ was also added, followed by 1.25 mL of each of 0.1M Yb(NO₃)₃.5H₂O and NH₄F solution with continuous stirring.
The mixture was left to stir for about 3 hours.
The mixture was left in a surrounding of about 25° C. to form a gel.

(6) Calcium Phosphate Doped with Cerium, Ytterbium and Fluorine (batch 1):
10 mL of tetraorthosilicate was added dropwisely to 25 mL of 0.1M Ca(NO₃)₂.4H₂O solution with continuous stirring.
11.25 mL of 0.1M (NH₄)₂HPO₄ was also added, followed by 1.25 mL of each of 0.1M Yb(NO₃)₃.5H₂O, Ce(NO₃)₃.6H₂O and NH₄F with continuous stirring.
The mixture was left to stir for about 3 hours.
The mixture was left in a surrounding of about 25° C. to form a gel. (7) Calcium Phosphate Doped with Cerium, Ytterbium and Fluorine (batch 2):
30 mL of tetraorthosilicate was added dropwisely to 75 mL of calcium nitrate solution with continuous stirring.
33.75 mL of 0.1M (NH₄)₂HPO₄ was also added, followed by 3.75 mL of each of 0.1M Yb(NO₃)₃.5H₂O, Ce(NO₃)₃.6H₂O and NH₄F with continuous stirring.
The mixture was left to stir for about 24 hours.
The mixture was left in a surrounding of about 25° C. for gelation to continue.

### EXAMPLE 4

A layer of the hydrogel formulation of the present invention was deposited prior to femtosecond laser irradiation at 1520 nm. The layer rapidly formed a smoother and more pristine surface than solely bioceramic material such as calcium phosphate phase-based materials. Irradiation followed by brushing trials demonstrated the benefits of densification of the calcium phosphate phases and its significance in providing wear resistance through rapid bonding and adhesion with the enamel dentine surface as shown in FIGS. 22a and b.

The hydrogel formulation is ideal for forming sprays or pastes for application to the surface of a tooth and may be cast into pre-fabricated mineral structures such as the hollow tube shown in FIG. 23. It may be possible to engineer the growth of glass or ceramic materials by controlling laser irradiation time and speed. X-ray diffraction spectra shown in FIGS. 24a and 24b confirms the onset of crystallisation in a hydrogel formulation which would lead to a progressive phase transformation to a composite structure in which porosity is also controlled by laser-induced densification.

### EXAMPLE 5

An embodiment of the hydrogel formulation of the invention which included chitosan was prepared as follows.

Step 1: 1 g of chitosan powder was dissolved in 100 ml of an aqueous lactic acid solution (2% v/v). Other acids can be also used (eg acetic acid). The mixture was stirred at 60° C. for 1 hour.

Step 2: NH₄F, Er(NO₃)₃.9H₂O and Sr(NO₃)₂ were added at concentrations between 0.1 and 0.5% w/v.

Step 3: 10 ml of tetraethyl orthosilicate was added in the amount tetraethyl orthosilicate:chitosan solution =1:10. The resulting mixture was stirred for 1 hour at 37° C. and thereafter for 3-5 days at room temperature until gelation took place.

Step 4: The chitosan/orthosilicate mixture was mixed with brushite crystals in the amount gel: brushite =10:1 by weight. The resulting hydrogel formulation was a non-Newtonian (shear thinning) suspension (see FIG. 25). The final viscosity which is critical for controlling the thickness of the coating could be adjusted by changing the ratio of gel: brushite.

Laser Treatment

The hydrogel formulation was applied to tooth enamel in a homogeneous thin layer (around 20 μm) and left to dry at room temperature for 10 minutes. Irradiation experiments were conducted with femtosecond pulsed lasers and continuous wave (CW) lasers. In both cases, melting of the brushite crystals and the formation of a remineralised surface were observed (see FIG. 26).

Advantages of the Use of Chitosan

The advantages of the addition of chitosan to the hydrogel formulation are threefold:
Results in compact coatings where the brushite crystals are homogeneously distributed while the porosity between them is reduced significantly. During laser irradiation this is very important since it contributes to effective heat dissipation and eventually to the sintering of the material.
The adhesion of the tetraethyl orthosilicate hydrogel formulation with chitosan on enamel and titanium samples was high compared with the tetraethyl orthosilicate hydrogel formulation without chitosan (see FIG. 27).
The presence of chitosan promotes cell proliferation and growth which is important for bone grafts or coatings for titanium implants.

1. A hydrogel formulation comprising:
a solid phase composed of a continuous network of siloxane bonds and one or more calcium phosphate phases doped with one or more metal dopants; and
an aqueous phase, or a precursor or anhydrate thereof.

2. The hydrogel formulation as claimed in claim 1 wherein the one or more metal dopants is or includes ions of a rare earth element.

3. The hydrogel formulation as claimed in claim 2 wherein the rare earth element is selected from the group consisting of erbium, cerium and ytterbium.

4. The hydrogel formulation as claimed in claim 1, wherein the one or more metal dopants is or includes aluminium ions.

5. The hydrogel formulation as claimed in claim 1, wherein the one or more calcium phosphate phases are fluoride ion-substituted.

6. The hydrogel formulation as claimed in claim 1, wherein the one or more calcium phosphate phases includes fluorapatite.

7. The hydrogel formulation as claimed in claim 1, wherein the one or more calcium phosphate phases are fluoride ion-substituted and the one or more metal dopants is or includes aluminium ions.

8. The hydrogel formulation as claimed in claim 1, wherein the one or more calcium phosphate phases are fluoride ion-substituted and the one or more metal dopants is or includes ions of a rare earth element.

9. The hydrogel formulation as claimed in claim 1, wherein the one or more calcium phosphate phases are fluoride ion-substituted and the one or more metal dopants is or includes aluminium ions and ions of a rare earth element.

10. The hydrogel formulation as claimed in claim 1, wherein the one or more calcium phosphate phases is or includes synthetic hydroxyapatite or a synthetic precursor thereof.

11. The hydrogel formulation as claimed in claim 1, wherein the one or more calcium phosphate phases is or includes a synthetic mineral of formula CaHPO₄.xH₂O (wherein x is 0, 1 or 2).

12. The hydrogel formulation as claimed in claim 1, wherein the one or more calcium phosphate phases include monetite which is the predominant calcium phosphate phase.

13. The hydrogel formulation as claimed in claim 1, wherein the one or more calcium phosphate phases include brushite which is the predominant calcium phosphate phase.

14. The hydrogel formulation as claimed in claim 1, wherein the solid phase is further composed of chitosan.

15. The hydrogel formulation as claimed in claim 1, obtained or obtainable by a process comprising:
(a) preparing an aqueous mixture of a calcium ion-containing solution, a phosphate ion-containing solution and a metal dopant-containing solution in the presence of the siloxane network precursor;
(b) causing the formation of the solid phase in the aqueous mixture; and
(c) isolating the hydrogel formulation.

16.-30. (canceled)
